(19) United States
(12) Patent Application Publication
(10) Pub. No.: US 2024/0117284 A1
(43) Pub. Date: Apr. 11, 2024

(54) EXTRACTION OF NUTRIENT SUPPLMENT PRODUCT USING ENZYME DIGESTION OF CELL MASS

(71) Applicant: Jupeng Bio (HK) Limited, Hong kong (CN)

(72) Inventors: Ryan H. Senaratne, Fayetteville, AR (US); Brandon Beard, Tontitown, AR (US)

(21) Appl. No.: 18/375,181

(22) Filed: Sep. 29, 2023

Related U.S. Application Data
(60) Provisional application No. 63/413,060, filed on Oct. 4, 2022.

(51) Int. Cl. C12M 1/00; A23L 33/135

(52) U.S. Cl. CPC C12M 21/00; A23L 33/135; C12M 23/34; C12M 23/40

(57) ABSTRACT

System and process are provided for producing and obtaining nutrient supplement products that are derived from microbial biomass from an anaerobic bacterial fermentation process using a myriad enzyme digestion and purification technique. More specifically, the disclosure provides process and system that simultaneously producing oxygenated hydrocarbon compounds at high specific productivity while effectively extracting nutrient supplement products from microbial biomass.

A process is provided for producing products, materials, intermediates, and the like such as organic acids, single cell protein, alcohols, and organic acid salts from a bacterial fermentation process. More specifically, the process includes recovering microbial cells from industrial fermentation process and extracting the cell mass by using enzyme digestion into single cell proteins to be used as nutrient supplements.

### BACKGROUND

Carbon monoxide and carbon dioxide emissions from industrial processes are two of the major drivers of climate change and global warming. Microbial fermentation can reduce such carbon emissions by utilizing microorganisms, through their metabolic pathways, to convert Cl-containing gaseous substrate into useful oxygenated hydrocarbon compounds, such as ethanol, butanol, acetate, butyrate, 2,3-butanediol, and other desired products.

Large scale microbial fermentation also produces large amount of microbial biomass. Traditionally, disposal of microbial biomass needs highly expensive waste treatment system, storage sites and landfills. Previously finding, shows microbial biomass can be recovered into single cell protein (SCP) and other components for reuse as source of proteins, amino acids, and carbohydrates that are useful as a nutrient supplement for animals, plants, or human beings. For example, U.S. Pat. No. 10,856,560 describes a method of producing whole cell animal feed by culturing acetogens to produce microbial biomass.

However, current methods of recovering microbial biomass often directly use microbial cells as whole cell biomass nutrient supplement and such whole cells may contain high nucleic acid content that is not suitable for digestion. Accordingly, there remains a need for a process and system for effectively converting microbial biomass into digestible nutrient supplements, and compositions of any such nutrient supplements.

### SUMMARY

In accordance with the present disclosure, system, process, and compositions are provided for effectively producing and obtaining nutrient supplement products that are derived from microbial biomass from an anaerobic bacterial fermentation process using a myriad enzyme digestion and purification technique. The nutrient supplement products can be used directly or together with other nutrients as supplements for human, animal, microorganism, or plant.

A process for producing a nutrient supplement from an acetogenic bacteria in an anerobic fermentation process is provided. The process includes fermenting a gaseous substrate with the acetogenic bacteria in a fermentation vessel. Liquid fermentation broth containing acetogenic bacterial cells is obtained and separated into a cell-free permeate and a cell-containing suspension. An oxygenated hydrocarbon compound is recovered from the cell-free permeate. Once the cell-containing suspension is obtained, the process further includes increasing the pH of the cell-containing suspension, contacting the cell-containing suspension having increased pH with a hydrolase enzyme, and incubating the cell-containing suspension and the hydrolase enzyme at a temperature of about 50 to about 70° C. for about 3 to 72 hours to form a hydrolyzed lysate. The process further includes separating the hydrolyzed lysate into a protein-containing supernatant and a solid cell debris portion.

A system for producing a nutrient supplement and an oxygenated hydrocarbon compound from a bacterial fermentation process using acetogenic bacteria is provided. The system includes a fermentation vessel containing culture medium and acetogenic bacteria connected to a gas inlet line for flowing a gaseous substrate into the fermentation vessel to ferment the gaseous substrate and the culture medium with the acetogenic bacteria to produce a fermentation liquid broth. The system includes one or more cell separators connected to one or more outlets of the fermentation vessel to receive the liquid fermentation broth and separate the liquid fermentation broth into a cell-free permeate and a cell-containing suspension. Once the cell-free permeate and the cell-containing suspension are produced, the system further includes a distillation chamber receiving the cell-free permeate and produces the oxygenated hydrocarbon compound, and a digestion tank connected to one or more outlet lines of the one or more cell separators to receive the cell-containing suspension and incubate the cell-containing suspension with hydrolase enzyme at an incubation temperature at about 50 to 70° C. to produce a hydrolyzed lysate. The system further includes one or more fractionators connected to one or more outlet lines of the digestion tank to receive the hydrolyzed lysate and produce a protein-containing supernatant and a cell debris portion.

A process for producing a nutrient supplement from an anaerobic fermentation process is provided. The process includes fermenting a gaseous substrate with a first acetogenic bacteria in a first fermentation vessel to produce a first vent gas and a first fermentation liquid broth containing the first acetogenic bacterial cells. The first fermentation liquid broth is separated into a first cell-free permeate and a first cell-containing suspension. An oxygenated hydrocarbon compound is recovered from the first cell-free permeate. The process includes fermenting at least a portion of the first vent gas with a second acetogenic bacteria in a second fermentation vessel to produce a second fermentation liquid broth containing the second acetogenic bacterial cells. The second fermentation liquid broth is separated into a second cell-free permeate and a second cell-containing suspension. At least a portion of the second cell-free permeate is recycled to the first fermentation vessel. Once the first cell-containing suspension and the second cell-containing suspension are produced, the process further includes blending at least a portion of the first cell-containing suspension with at least a portion of the second cell-containing suspension to form a mixed cell-containing suspension, increasing the pH of the mixed cell-containing suspension, contacting the mixed cell-containing suspension having increased pH with a hydrolase enzyme, and incubating the mixed cell-containing suspension and the hydrolase enzyme at a temperature of about 50 to about 70° C. for about 3 to 72 hours to form a hydrolyzed lysate. The process further includes separating the hydrolyzed lysate into a protein-containing supernatant and a solid cell debris portion.

A process for producing a nutrient supplement from an anaerobic fermentation process is provided. The process includes fermenting a gaseous substrate with a first acetogenic bacteria in a first fermentation vessel to produce a first vent gas and a first fermentation liquid broth containing the first acetogenic bacterial cells. Further, at least a portion of the first vent gas is fermented with a second acetogenic bacteria in a second fermentation vessel to produce a second fermentation liquid broth containing the second acetogenic bacterial cells. At least a portion of the first fermentation liquid broth containing the first acetogenic bacterial cells and at least a portion of the second fermentation liquid broth containing the second acetogenic bacterial cells are blended to form a mixed fermentation liquid broth. The process further includes separating the mixed fermentation liquid broth to produce a cell-free permeate and a cell-containing suspension. An oxygenated hydrocarbon compound is recovered from the cell-free permeate. Once the cell-containing suspension is produced, the process further includes increasing the pH of the cell-containing suspension, contacting the cell-containing suspension having increased pH with a hydrolase enzyme, incubating the cell-containing suspension and hydrolase enzyme at a temperature of about 50 to about 70° C. for about 3 to 72 hours to form a hydrolyzed lysate, and fractionating the cell-containing suspension into a protein-containing supernatant and a solid cell debris portion.

A process for producing a nutrient supplement from an anaerobic fermentation process is provided. The process includes fermenting a gaseous substrate with a first acetogenic bacteria in a first fermentation vessel to produce a first vent gas and a first fermentation liquid broth containing the first acetogenic bacterial cells. The first fermentation liquid broth is separated into a first cell-free permeate and a first cell-containing suspension. An oxygenated hydrocarbon compound is recovered from the first cell-free permeate. The process includes fermenting at least a portion of the first vent as with a second acetogenic bacteria in a second fermentation vessel to produce a second fermentation liquid broth containing the second acetogenic bacterial cells. The second fermentation liquid broth is separated into a second cell-free permeate and a second cell-containing suspension. At least a portion of the second cell-free permeate is recycled to the first fermentation vessel. Once the first cell-containing suspension is produced, the process further includes increasing the pH of the first cell-containing suspension, contacting the first cell-containing suspension having increased pH with hydrolase enzyme, incubating the first cell-containing suspension and hydrolase enzyme at a temperature of about 50 to about 70° C. for about 3 to 72 hours to form a hydrolyzed lysate, and fractionating the first cell-containing suspension into a first protein-containing supernatant and a first solid cell debris portion. Once the second cell-containing suspension is produced, the process further includes increasing the pH of the second cell-containing suspension, contacting the second cell-containing suspension having increased pH with a hydrolase enzyme, incubating the second cell-containing suspension and hydrolase enzyme at a temperature of about 50 to about 70° C. for about 3 to 72 hours to form a hydrolyzed lysate, and fractionating the second cell-containing suspension into a second protein-containing supernatant and a second solid cell debris portion.

A process for producing a nutrient supplement from an acetogenic bacteria in an anerobic fermentation process is provided. The process includes fermenting a gaseous substrate with the acetogenic bacteria in a fermentation vessel. Liquid fermentation broth containing acetogenic bacterial cells is obtained and separated into a cell-free permeate and a cell-containing suspension. An oxygenated hydrocarbon compound is recovered from the cell-free permeate. Once the cell-containing suspension is obtained, the process further includes increasing the pH of the cell-containing suspension, contacting the cell-containing suspension having increased pH with a hydrolase enzyme, and incubating the cell-containing suspension and the hydrolase enzyme at a temperature of about 50 to about 70° C. for about 2 to 36 hours to form a partial hydrolyzed lysate. The partial hydrolyzed lysate is then mechanically ruptured into a hydrolyzed lysate. The process further includes separating the hydrolyzed lysate into a protein-containing supernatant and a solid cell debris portion.

### BRIEF DESCRIPTION OF FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a schematic of a system for producing a protein-containing supernatant and one or more oxygenated hydrocarbon compounds from a fermentation process using one species of acetogenic bacteria.

FIG. 2 illustrates a schematic of a system for producing a protein powder and one or marc oxygenated hydrocarbon compounds from a fermentation process using one species of acetogenic bacteria.

FIG. 3 illustrates a schematic of a system for producing an amino acid fertilizer and one or more oxygenated hydrocarbon compounds from a fermentation process using one species of acetogenic bacteria.

FIG. 4 illustrates a schematic of a system for producing a protein-containing supernatant and one or more oxygenated hydrocarbon compounds from a multi-vessel fermentation process using two or more species of acetogenic bacteria.

FIG. 5 illustrates a schematic of a system for producing a protein powder and one or more oxygenated hydrocarbon compounds from a multi-vessel fermentation process using two or more species of acetogenic bacteria.

FIG. 6 illustrates the growth of Escherichia coli (E. coli) with protein containing supplement as microbial nutrition.

### DETAILED DESCRIPTION

The following description is not to be taken in a limiting sense but is made merely for the purpose of describing the general principles of exemplary embodiments. The scope of the disclosure should be determined with reference to the claims.

The term “about” modifying any amount refers to the variation in that amount encountered in real world conditions, e.g., in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in multiple experiments in the plant or lab and the variation inherent in the analytical method. Whether or not modified by “about” the amounts include equivalents to those amounts. Any quantity stated herein and modified by “about” can also be employed in the present disclosure as the amount not modified by “about”.

The use of the terms “a”, “an”, “the” and similar referents in the context of this disclosure are to be construed to cover both the singular and the plural, unless otherwise indicated or clearly contradicted by context.

Unless otherwise indicated, the terms “comprising”, “including,” “having”, “containing”, or “characterized by” are inclusive and does not exclude any additional, unrecited elements or method steps (i.e., meaning “including, but not limited to”). The use of any and all examples or exemplary language (e.g., “such as”, “for example”, “for instance”) provided herein is intended merely to illuminate the disclosure and does not impose a limitation on the scope of the disclosure unless otherwise claimed.

Fermentation is a metabolic process used by bacteria to generate energy for cell growth. Certain anaerobic bacteria are capable of fermenting a Cl-containing gaseous substrate, such as CO-containing gaseous substrate and CO2-containing gaseous substrate, to sustain their growth and produce oxygenated hydrocarbon compounds. These anaerobic bacteria may use the carbon from the Cl-containing gaseous substrate as the only carbon source for their growth during the fermentation process. The terms “fermentation”, “fermentation process”, “microbial fermentation process” and the like are intended to encompass both the growth phase and the product biosynthesis phase of the process. During an anaerobic bacterial fermentation process, large amounts of microbial biomass are obtained, which may be purged out and processed to useful products, such as nutrient supplements. Specifically, the present disclosure includes a process of extracting nutrient supplements out of microbial biomass from an anaerobic fermentation process.

Anaerobic bacteria are bacteria that do not require oxygen for growth. An anaerobic bacteria may react negatively or even die if oxygen is present above a certain threshold. Acetogenic bacteria are microorganisms that are capable of producing acetate under anaerobic respiration or fermentation by utilizing the Wood-Ljungdahl pathway as their main mechanism for energy conservation. Other useful oxygenated hydrocarbon compounds, such as formic acid, propionic acid, butyric acid, heptanoic acid, decanoic acid, ethanol, butanol, 2-butanol, and 2,3-butanediol, may also be produced by the acetogenic bacteria. Examples of the acetogenic bacteria suitable for converting Cl-containing gaseous substrate to useful oxygenated hydrocarbon compounds include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 200208438, strains of Clostridium autoethanogenum (DSM 10061 and DSM 1063) of DSMZ, Germany) including those described in WO 2007/117157 and WO) 2009/151342 and Clostridium ragsdalei (P11, ATCC BAA-622) and Alkalibaculum bacchi (CP11, ATCC BAA-1772) including those described respectively in U.S. Pat. No. 7,704,723 and “Biofuels and Bioproducts from Biomass-Generated Synthesis Gas”, Hasan Atiyeh, presented in Oklahoma EPSCoR Annual State Conference, Apr. 29, 2010 and Clostridium carboxldivorans (ATCC PTA-7827) described in U.S. Patent Application No. 2007/0276447. Other suitable microorganisms include those of the genus Moorella, including Moorella sp. HUC22-1, and those of the genus Carboxydothermus. Each of these references is incorporated herein by reference. Mixed cultures of two or more microorganisms may also be used.

Additional examples of useful acetogenic bacteria include Acetogenium kivui, Acetoanaerobium noterae, Acetobacterium woodii, Alkahbaculum bacchi CP11 (ATCC BAA-1772), Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clotridium acetobutylicum P262, Clostridium autoethanogenum (DSM 19631 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans P7 (ATCC PTA-7827), Clostridium coskatii (ATCC PTA-1052), Clostridium drakei, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdelei P11 (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Moorella thermoacetica, Moorella thermoautotrophica, Oxobacter pfennigii, Peptostreptococcus productus, Ruminococcus productus, Thermoanaerobacter kivui, Clostridium Stick-landii, and mixtures thereof.

Fermentable gaseous substrate refers to Cl-containing gaseous substrate comprises one or more of CO or CO₂. Suitable gaseous substrate may include various synthesis gas (i.e., syngas) and industrial off-gas.

Syngas may be provided from any known source. In one aspect, syngas may be sourced from gasification of carbonaceous materials. Gasification involves partial combustion of biomass in a restricted supply of oxygen. The resultant gas may include CO, CO₂, and H₂. Some examples of suitable gasification methods and apparatus are provided in U.S. Ser. Nos. 61/516,667, 61/516,667, 64/516,704 and 61/516,646, all of which were fled on Apr. 6, 2011, and in U.S. Ser. Nos. 13/427,144, 13/427,193 and 13/427,247, all of which were filed on Mar. 22, 2012, and all of which are incorporated herein by reference. In another aspect, syngas may be generated from electrolysis of water and carbon dioxide. In this aspect, oxygen is removed from the resultant gas and the resultant gas may be further blended with other gas sources to form a desired fermentables gaseous substrate.

Industrial off-gas may include the Cl-containing waste gas from industrial processes that would otherwise be exhausted into the atmosphere. Examples of industrial off-gas include gases produced during microbial fermentation, ferrous metal products manufacturing, non-ferrous products manufacturing, petroleum refining processes, gasification of coal, electric power production, carbon black production, ammonia production, methanol production, coke manufacturing and gas reforming.

The Cl-containing gaseous substrate may include H₂, H₂ may also be separately supplemented into the Cl-containing gaseous substrate to form desired gas composition suitable for fermentation. Examples of H₂ source include gases produced during ferrous metal products manufacturing, non-ferrous products manufacturing, petroleum refining processes, gasification of coal, gasification of biomass, electric power production, carbon black production, ammonia production, methanol production and coke manufacturing. Other sources of hydrogen may include for example, H₂O electrolysis and bio-generated H₂.

The fermentation of the fermentable gaseous substrate with the acetogenic bacteria takes place in a fermentation vessel. Fermentation vessel includes a fermentation bioreactor consisting of one or more vessels and/or towers or piping arrangements, which includes a batch reactor, semi-batch reactor, continuous reactor, continuous stirred tank reactor (CSTR) bubble column reactor, external circulation loop reactor, internal circulation loop reactor, immobilized cell reactor (ICR), trickle bed reactor (TBR), moving bed biofilm reactor (MBBR), gas lift reactor, membrane reactor such as hollow fiber membrane bioreactor (HFMBR), static mixer, gas lift fermentor, or other vessel or other device suitable for gas-liquid contact.

A culture medium suitable for anaerobic bacterial growth and fermenting fermentable gaseous substrate into one or more oxygenated hydrocarbon compounds can be added to the fermentation vessel to support the fermentation of the gaseous substrate by the acetogenic bacteria. Some examples of medium compositions are described in U.S. Ser. Nos. 16/530,501 and 16/530,481, filed Aug. 2, 2019, and in U.S. Pat. No. 7,285,402, filed Jul. 23, 2001, all of which are incorporated herein by reference. The medium may be sterilized to remove undesirable microorganisms and the fermentation vessel is inoculated with the desired microorganisms. Sterilization may not always be required.

The fermentation process of the underlying disclosure provides a simultaneous approach of generating a high specific productivity of oxygenated hydrocarbon compound production while producing nutrient supplement from the bacterial cells used m the fermentation process. As used herein, specific productivity is expressed as specific STY. In this aspect, specific oxygenated hydrocarbon compound productivity may be expressed as specific STY (e.g. specific space time yield can be expressed as & alcohol/day/gram of cells or g organic acid/day/gram of cells). In one aspect, the fermentation process provides a specific organic acid productivity of about 0.2 to about 100 rams organic acid/day/gram of cells, in another aspect, about 0.2 to about 70 grams organic acid/day/gram of cells, in another aspect, about 0.2 to about 50 grams organic acid/day/grain of cells, in another aspect, about 0.2 to about 20 grams organic acid/day/gram of cells, in another aspect, about 10 to about 50 grams organic acid/day/gram of cells, in another aspect, about 12 to about 30 grams organic acid/day/gram of cells, in another aspect, about 2 to about 20 grains organic acid/day/gram of cells, in another aspect, about 15 to about 35 grams organic acid/day/gram of cells, and in another aspect, about 25 to about 70 grams organic acid/day/gram of cell. In this aspect, the organic acid is acetic acid or butyric acid, or a mixture of both. In another aspect, the fermentation process provides a specific alcohol productivity of about 10 grams alcohol/day/grams of cells or more, in another aspect, a specific alcohol productivity rate of about 12 g/day/grams of cells or more, in another aspect, a specific alcohol productivity rate of about 14 g/day/grams of cells or more, in another aspect, a specific alcohol productivity rate of about 11 to about 16 g/day/grams of cells, in another aspect, about 10 to about 14 g/day/grams of cells, in another aspect, about 10 to about 12 g/day/grams of cells, in another aspect, about 10 to about 16 g/day/grans of cells, in another aspect, about 10 to about 14 g/day/grams of cells, in another aspect, about 12 to about 16 g/day/grams of cells, and in another aspect, about 12 to about 14 g/day/grams of cells. In this aspect, the alcohol is ethanol or butanol, or a mixture of both.

Further, the fermentation process can be manipulated under conditions that facilitate the production of desired product. In one aspect, the desired product is one or more oxygenated hydrocarbon compounds. In another aspect, the desired product is the microbial biomass itself, and the process also produces other oxygenated hydrocarbon compounds as byproducts. Operation parameters, such as culture medium flow rate, gaseous substrate feed rate, water supply/recycle rate, temperature, media redox potential, pressure. pH, agitation rate (if using a stirred tank reactor), and cell concentration, are monitored and controlled throughout the fermentation process.

A fermentation liquid broth is generated inside the fermentation vessel once the fermentation process starts. In addition to the culture medium, the fermentation liquid broth also includes acetogenic bacteria and one or more oxygenated hydrocarbon compounds. In one aspect, the cell concentration of the fermentation liquid broth is about 1 to about 15 g/L, in another aspect 2 to about 30 g/L, in another aspect, about 2 to about 25 g/L, in another aspect, about 2 to about 20 g/L, in another aspect, about 2 to about 10 g/L, in another aspect, about 2 to about 8 g/L, in another aspect, about 3 to about 30 g/L, in another aspect, about 3 to about 6 g/L. and in another aspect, about 4 to about 5 g/L.

The fermentation liquid broth is further purged out of the fermentation vessel and then separated into a cell-free permeate and a cell-containing suspension by one or more cell separators. Suitable cell separators include, but not limited to, filtration devices, hollow fiber filtration devices, spiral wound filtration devices, ultrafiltration devices, ceramic filter devices, cross-flow filtration devices, size exclusion column filtration devices, spiral wound membranes, centrifugation devices, and combination thereof.

The cell-free permeate contains one or more desired oxygenated hydrocarbon compounds and is sent to a distillation chamber for product recovery. One or more desired product is recovered and collected from the distillation chamber. In one aspect, a holding tank is placed between the one or more cell separator and the distillation chamber to receive the cell-free permeate and control cell-free permeate flow rate to the distillation chamber. Distillation bottoms may be recycled back to the fermentation vessel. In one aspect, at least a portion of the distillation bottoms is recycled back to the fermentation vessel. In another aspect, at least a portion of the distillation bottoms is sent to a wastewater treatment system for further treatment in still another aspect, at least a portion of the distillation bottoms is recycled back to the fermentation vessel and at least a portion of the distillation bottoms is sent to a wastewater treatment system.

The cell-containing suspension contains acetogenic bacterial cells at a cell concentration higher than the fermentation liquid broth. In one aspect, the cell concentration of the cell-containing suspension is about 20 g/L or more, in another aspect, about 0.30 g/L or more, in another aspect, about 40 g/L or more. in another aspect, about 50 g/L or more, in another aspect, about 60 g/L or more, in another aspect, about 20 to about 300 g/L, in another aspect, about 30 to about 250 g/L, in another aspect, about 40 to about 200 g/L, in another aspect, about 50 to about 150 g/L, in still another aspect, about 100 to about 150 g/L. The cell-containing suspension may be recycled back to the fomentation vessel to maintain and control the cell concentration in the fermentation process. Additional cell-containing suspension can also be further processed into nutrient supplement. In one aspect, at least a portion of the cell-containing suspension is recycled back to the fermentation vessel. In another aspect, at least a portion of the cell-containing suspension is further processed to nutrient supplement. In still another aspect, at least a portion of the cell-containing suspension is recycled back to the fermentation vessel and at least a portion of the cell-containing suspension is further processed to nutrient supplement.

Multiple cell separators may be used in the fermentation process to adjust and balance the production of the oxygenated hydrocarbon compound and the nutrient supplement so that desired productivity of oxygenated hydrocarbon compound is maintained while bacterial cells are produced into nutrient supplement. In one aspect, at least two cell separators are used. In this aspect, a first fermentation liquid broth is sent to a first cell separator to produce a first cell-free permeate and a first cell-containing suspension. The first cell-free permeate is further sent to a distillation chamber for the production of oxygenated hydrocarbon compound and at least a portion of the first cell-containing suspension is recycled back to the fermentation vessel to control and maintain cell concentration of the fermentation liquid broth. Further, a second fermentation liquid broth is sent to a second cell separator to produce a second cell-free permeate and a second cell-containing suspension. The second cell-free permeate is sent to the distillation chamber for the production of oxygenated hydrocarbon compound and the second cell-containing suspension is then processed into nutrient supplement. In another aspect, three or more cell separators are used in a multi-vessel fermentation process.

### CO Bioconversion

Acetogenic bacteria can ferment CO-containing gaseous substrate into useful oxygenated hydrocarbon compounds, such as ethanol and butanol. In this aspect, suitable gaseous substrate obtains at least about 10 mole % CO, in one aspect, at least about 20 mole %, in one aspect, at least about 30 mole %, in one aspect, about 10 to about 100 mole %, in another aspect, about 20 to about 100 mole % CO, in another aspect, about 30 to about 90 mole % CO, in another aspect, about 40 to about 80 mole % CO, and n another aspect, about 50 to about 70 mole % CO. In this aspect, the CO-containing gaseous substrate may have about 40 mole % or less CO₂, in one aspect, the CO-containing gaseous substrate may have about 30 mole % or less CO₂, in one aspect, the CO-containing gaseous substrate may have about 20 mole % or less CO₂, in another aspect, the CO-containing gaseous substrate may have about 10 mole % or less CO₂, in another aspect, the CO-containing gaseous substrate may have about 1 mole % or less CO₂, in still another aspect, the CO-containing gaseous substrate may comprise no or substantially no CO₂.

Depending on the composition of the CO-containing gaseous substrate, the CO-containing gaseous substrate may be directly provided to a fermentation process or may be further modified or blended to include an appropriate H₂ to CO molar ratio. In one aspect, the CO-containing gaseous substrate provided to the fermentation vessel has an H₂ to CO molar ratio of about 0.2 or more, in another aspect, about 0.25 or more, and in another aspect, about 0.5 or more.

Concentrations of various medium components for use in the CO bioconversion fermentation process are as follows:

| | Concentration | Feed Rate |
|---|---|---|
| Element | mg/L | μg/gram cells/min |
| NH₄⁺ | 164-6560 | 41-1640 |
| Fe | 1.7-68 | 0.425-17 |
| Ni | 0.07-2.81 | 0.017-0.702 |
| Co | 0.037-1.49 | 0.009-0.373 |
| Se | 0.027-1.1 | 0.006-0.274 |
| Zn | 0.116-4.64 | 0.198-5.95 |
| W | 0.8-32.1 | 0.26-8.03 |
| K | 39-1573 | 9.83-393.25 |
| Mg | 1.4-57.3 | 0.35-14.32 |
| S | 15-625 | 3.9-156.2 |
| P | 15-601 | 3.76-150.43 |
| d-biotin | 0.016-0.64 | 0.004-0.16 |
| thiamine HCl | 0.04-1.6 | 0.01-0.4 |
| calcium-D-pantothenate | 0.02-0.81 | 0.005-0.202 |

Examples of useful acetogenic bacteria for CO bioconversion fermentation process include Acetogenium kivui, Acetoanaerobium noterae, Acetobacterium woodii, Alkalibaculum bacchi CP11 (ATCC BAA-1772), Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium autoethanogenum P7 (ATCC PTA-7827), Clostridium coskatii (ATCC PTA-10522), Clostridium drakei, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Desulfotomaculum kuznetsovii, Eubacterium limosum, Geobacter sulferreducens, Methanosarcina acetivorans, Methanosarcina barkeri, Moorella thermoacetica, Moorella thermoautotrophica, Oxobacter pfennigii, Peptostreptococcus productus, Ruminococcus productus, Thermoanaerobacter kivui, Clostridium Stick-landii, and mixtures thereof.

The pH value of the fermentation broth for CO bioconversion is maintained in a range of about 3.5 to about 6.9, in one aspect, about 4 to about 6, in another aspect, about 4 to 5, in another aspect, about 4.2 to 4.8, another aspect, about 4.2 to 4.6, and in another aspect, about 4.4 to 4.8.

### CO₂ Bioconversion

Acetogenic bacteria may ferment CO₂-containing gaseous substrate into useful oxygenated hydrocarbon compounds, such as acetic acid and butyric acid. In one aspect, suitable CO₂-containing gaseous substrate contains at least about 10 mole CO₂, in one aspect, at least about 20 mole %, in one aspect, at least about 30 mole %, in one aspect, at least about 40 mole %, in one aspect, about 10 to about 70 mole %, in another aspect, about 20 to about 70 mole % CO₂, in another aspect, about 30 to about 70 mole % CO₂, in another aspect, about 40 to about 70 mole % CO₂ in another aspect, about 10 to about 50 mole % CO₂, in another aspect, about 20 v, about 40 mole % CO₂, and in still another aspect, about 30 to 50 mole % CO₂. In this aspect, the CO₂-containing gaseous substrate contains about 50 mole % or less CO, in one aspect, the CO₂-containing gaseous substrate contains about 40 mole 7% or less CO, in one aspect, the CO₂-containing gaseous substrate contains about 30 mole % or less CO, in one aspect, the CO-containing gaseous substrate contains about 20 mole % or less CO, in one aspect, the CO₂-containing gaseous substrate contains about 10 mole % or less CO, in one aspect, the CO₂-containing gaseous substrate contains about 5 mole % or less CO₂ in one aspect, the CO₂-containing gaseous substrate contains about 1 mole % or less CO, in another aspect, the CO₂-containing gaseous substrate contains no or substantially no CO.

Depending on the composition of the CO₂-containing gaseous substrate, the CO₂-containing gaseous substrate may be directly provided to a fermentation process or may be further modified or blended to include an appropriate H₂ to CO₂ molar ratio. For example, a stream comprising a high concentration of CO₂, such as the exhaust from an industrial process, can be combined with a stream comprising high concentrations of H₂, such as the off gas from a coke oven. In one aspect, the gaseous substrate provided to the fermentation vessel has an H₂ to CO₂ molar ratio of about 4:1 to about 1:2, in another aspect, about 4:1 to about 1:1, in another aspect, about 4:1 to about 2:1, and in still another aspect, about 3.5:1 to about 1.5:1.

Concentrations of various medium components for use in the CO₂ bioconversion fermentation process are as follows:

| | Concentration | Feed Rate |
|---|---|---|
| Element | mg/L | μg/gram cells/min |
| NH₄⁺ | 82-3280 | 20.5-820 |
| Fe | 0.85-34 | 0.28-8.5 |
| Ni | 0.07-2.81 | 0.023-0.702 |
| Co | 0.037-1.49 | 0.012-0.373 |
| Se | 0.027-1.1 | 0.009-0.274 |
| Zn | 0.59-23.8 | 0.198-5.95 |
| Mo | 0.003-0.397 | 0.003-0.1 |
| chelator | 2.5-100 | 0.83-25 |
| W | 0.8-32.1 | 0.26-8.03 |
| K | 98-3933 | 32.77-983.35 |
| Mg | 0.71-28.69 | 0.23-7.18 |
| Na | 875-35000 | 290-8750 |
| S | 15-625 | 2.08-62.5 |
| P | 20-805 | 6.7-201.3 |
| d-biotin | 0.016-0.64 | 0.005-0.16 |
| thiamine HCl | 0.04-1.6 | 0.01-0.4 |
| calcium-D-pantothenate | 0.02-0.81 | 0.006-0.202 |

Suitable acetogenic bacteria for CO₂ bioconversion may include a sodium pump which may also be described as sodium-translocating ATPases (for membrane bioenergetics). Sodium translocating ATPase are described in Muller, “Energy Conservation in Acetogenic Bacteria,” Appl. Environ. Microbiol. November 2003, vol. 69, no. 11, pp. 6345-6353, which is incorporated herein by reference. Acetogenic bacteria that include a sodium-translocating ATPase require about 500 ppm NaCl in their growth medium for growth. To determine if an acetogenic bacteria includes a sodium-translocating ATPase, the acetogen is inoculated into serum bottles containing about 30 to about 50 ml of growth medium with about 0 to about 2000 ppm NaCl. Normal growth at NaCl concentrations of about 500 ppm or more means that the acetogenic bacteria includes a sodium-translocating ATPase. In this aspect, the composition of the fermentation medium also includes a sodium ion concentration of about 40 to about 500 mmol per liter, in another aspect, about 40 to about 250 mmol per liter and in another aspect, a sodium ion concentration of about 50 to about 200 mmol per liter. In one aspect, the sodium ion concentration is about 500 ppm to about 8000 ppm, in another aspect, about 1000 ppm to about 7000 ppm, in another aspect, about 3000 ppm to about 6000 ppm, in another aspect, about 2000 to about 5000 ppm, and in another aspect, about 3000 to about 4000 ppm.

Examples of useful acetogenic bacteria for CO₂ bioconversion includes Acetogenium kivui, Acetoanaerobium noterae, Acetobacterium woodii, Alkalibaculum bacchi CP11, Moorella thermoacetica, Moorella thermoautotrophica, Ruminococcus productus, Acetogenium kivui, and combinations thereof.

The pH value of the fermentation broth for CO₂ bioconversion is set at a first pH value at the start of the inoculation and is gradually increased to a second pH value higher than the first pH value during the steady state. In one aspect, the first pH value is at or below 5.5 and the second pf value is at or above 6. In another aspect, the first pH value is at or below 5.8 and the second pH value is at or above 6. In another aspect, the first pH value is at or below 6 and the second pH value is at or above 6.2. In still another aspect, the first pH value is at or below 6 and the second pH value is at or above 6.5.

### CO and CO₂ Bioconversion

The fermentation process may include two or more fermentation vessels to conduct both CO bioconversion and CO₂ conversion. For example, the fermentation process may contain one or more first fermentation vessels with a first acetogenic bacteria for CO bioconversion and one or more second fermentation vessels with a second acetogenic bacteria for CO₂ bioconversion. In this aspect, the first acetogenic bacteria and the second acetogenic bacteria are from different species. Vent gas from CO bioconversion contains CO₂ and can be used in the one or more subsequent CO bioconversion fermentation vessels as a CO₂-containing gaseous substrate. One or more fermentation liquid broth streams are purged out of the two or more fermentation vessels and separated into one or more one or more cell-free permeates and cell-containing suspensions through one or more cell separators. One or more oxygenated hydrocarbon compound can later be recovered from the one or more cell-free permeate. Meanwhile, the permeate containing the oxygenated hydrocarbon compound produced in CO₂ bioconversion process is sent to the one or more CO bioconversion fermentation vessels and the said oxygenated hydrocarbon compound may be fermented into another oxygenated hydrocarbon compound. In one aspect, the oxygenated hydrocarbon compound produced in CO₂ bioconversion is acetic acid. In this aspect, the permeate containing acetic acid is further delivered to the one or more CO bioconversion fermentation vessels and acetic acid is subsequently fermented into ethanol.

At least a portion of the one or more cell-containing suspensions is further processed into nutrient supplement. In one aspect, the at least a portion of the one or more cell-containing suspensions are mixed to form a mixed cell-containing suspension. The mixed cell-containing suspension has acetogenic bacterial cells from different species and is further processed into nutrient supplement. In another aspect, the one or more cell-containing suspensions are not blended and are separately processed into nutrient supplements. In still another aspect, at least a portion of the one or more fermentation liquid broth from the two or more fermentation vessels are sent into the same cell separator. In this aspect, a mixed cell-containing suspension has acetogenic bacterial cells from different species is delivered out from the cell separator and is further processed into nutrient supplement.

Nutrient Supplement Processing from Microbial Biomass

The at least a portion of the cell-containing suspension collected from the one or more cell separators can further be processed into nutrient supplement. The cell-containing suspension is continuously or intermittently sent to a digestion tank for enzyme hydrolysis. Enzyme is used to hydrolyze cell membrane and release inter-cellular materials, such as proteins, amino acids, metals (e.g., Ca, Cl, Co, K, Mg, Ni P, S, Se, W, Zn, Na, Fe), lipids, nucleic acids, and sugar. Suitable hydrolase enzyme includes protease, subtilases, alcalase, serine protease, serine endopeptidase, and combinations thereof.

Adjusting the pH value of the cell-containing suspension can facilitate enzyme hydrolysis. Treating cells with pH-adjusting agents makes the cell membrane more malleable to hydrolase enzyme. Further, the pH value also affects the activity of enzyme. Typically, each enzyme works best at a specific pH value or pH value range. In one aspect, the pH value suitable for enzyme hydrolysis is a pH of 7 or greater, in one aspect, a pH of 7.5 or greater, in one aspect, a pH of 8 or greater, in one aspect, a pH of 7 to 12, in one aspect, a pH of 7 to 11, in one aspect, a pH of 7 to 10, in one aspect, a pH of 7 to Q, in one aspect, a pH of 8 to 11, in one aspect, a pH of 8 to lii, and in one aspect, a pH of 8 to 9. The pH-adjusting agent added can be any acid, base, or salt. For example, suitable pH-adjusting agents include sodium hydroxide, potassium hydroxide, ammonium hydroxide, bicarbonate, hydrochloric acid, nitric acid, hydrogen chloride, and combinations thereof. In one aspect, the pH-adjusting agent is directly added to the digestion tank. In another aspect, the pH-adjusting agent is added before the cell-containing suspension enters the digestion tank. In one particular aspect, the cell-containing suspension has an acidic pH and the pH-adjusting agent added is a base.

Temperature of the ceil-containing suspension and processing time can also affect the efficiency of enzyme hydrolysis. Therefore, a temperature control unit can be installed to adjust, control, and maintain the temperature of the digestion tank. In one aspect, the temperature of the digestion tank is adjusted to about 40° C. to about 80° C., in one aspect, about 45° C. to about 75° C., in one aspect, about 50° C. to about 70° C., in one aspect, about 55° C. to about 65° C., in one aspect, about 70° C., in one aspect, about 65° C., in one aspect, about 60° C. In one aspect, the processing time of the enzyme hydrolysis is about 3 to 72 hours, in another aspect, about 5 to 60 hours, in another aspect, about 5 to 48 hours, in another aspect, about 12 to 36 hours, in another aspect, about 16 to 30 hours, in another aspect, about 20 to 25 hours, in another aspect, about 5 hours, in another aspect, about 12 hours, in another aspect, about 16 hours, in another aspect about 24 hours, in another aspect, about 36 hours, and in another aspect, about 48 hours.

The digestion tank may also include an agitator. In one aspect, the agitator provides an agitation rate of about 100 rpm or more, in one aspect, about 150 rpm or more, in one aspect, about 200 rpm or more, in one aspect, about 250 rpm or more, in one aspect, about 300 rpm or more, in another aspect, about 150 to about 1000 rpm, in another aspect, about 200 to about 800 rpm, in another aspect, about 350 to about 650 rpm, and in still another aspect, about 300 to about 450 rpm.

Hydrolyzed lysate is formed after enzyme hydrolysis. The hydrolyzed lysate is then delivered to one or more fractionators and fractionated into a protein-containing supernatant and a solid cell debris portion. Suitable fractionators for fractionating the hydrolyzed lysate includes, but not limited to, centrifuge devices, decanter centrifuges, disc-stack centrifuges, filtration devices, hollow fiber filtration devices, spiral wound filtration devices, ceramic filter devices, cross-flow filtration devices, size exclusion devices, exchange columns, carbon polymer columns, and combinations thereof. In one aspect, at least one fractionator is a centrifuge. In another aspect, at least one fractionator is an ultrafiltration device. In this aspect, the ultrafiltration device may be a 20 kDa to 60 kDa ultrafilter, in one aspect, a 100 kDa to 500 kDa ultrafilter, and in one aspect, a 3.00 kDa to 500 kDa ultrafilter. In another aspect, the ultrafiltration device may be a 0.05 to 0.4 μm ultrafilter, in one aspect, a 0.1 to 0.3 μm ultrafilter, and in one aspect, a 0.1 to 0.2 μm ultrafilter.

Alternatively, a partial hydrolyzed lysate is formed after a shortened period of enzyme hydrolysis. In this aspect, the shortened processing rime of the enzyme hydrolysis in the digestion tank is about 2 to about 36 hours, in one aspect, about 3 to about 24 hours, in one aspect, about 3 to 12 hours, in one aspect, about 4 to 10 hours, in one aspect, about 3 to 7 hours, in another aspect, about 3 hours, in another aspect, about 4 hours, and in still another aspect, about 5 hours. The partial hydrolyzed lysate is then sent to a mechanical rupturing device to be further ruptured into a hydrolyzed lysate. Suitable mechanical rupturing device includes, but not limited to, microfluidizer, sonication device, ultrasonic device, and French press. Since the partial hydrolyzed lysate is formed from the shortened period of enzyme hydrolysis in the digestion tank, the energy input of the mechanical rupturing device is significantly lower than the energy used in rupturing the cells of a non-hydrolyzed cell-containing suspension. A hydrolyzed lysate is then formed through mechanical rupturing and delivered to one or more fractionators to be fractionated into a protein-containing supernatant and a solid cell debris portion. Suitable fractionators for fractionating the hydrolyzed lysate includes, but not limited to, centrifuge devices, decanter centrifuges, disc-stack centrifuges, filtration devices, hollow fiber filtration devices, spiral wound filtration devices, ceramic filter devices, cross-flow filtration devices, size exclusion devices, exchange columns, carbon polymer columns, and combinations thereof. In one aspect, at least one fractionator is a centrifuge. In another aspect, at least one fractionator is an ultrafiltration device. In this aspect, the ultrafiltration device may be a 20 kDa to 600 kDa ultrafilter, in one aspect, a 100 kDa to 500 kDa ultrafilter, and in one aspect, a 300 kDa to 500 kDa ultrafilter. In another aspect, the ultrafiltration device may be a 0.05 to 0.4 μm ultrafilter, in one aspect, a 0.1 to 0.3 μm ultrafilter, and in one aspect, a 0.1 to 0.2 μm ultrafilter.

The solid cell debris portion may be directly used as or further processed to a nutrient supplement. In one aspect, the solid cell debris portion contains about 8 to about 30% of protein, in another aspect, about 8 to about 20% of protein, and in another aspect, about 8 to about 16% of protein. A dehydration unit may be used to dry the solid cell debris portion into low moisture content and the dried solid cell debris portion can be blended with other ingredients for making into one or more types of nutrient supplements. Suitable dehydration unit includes spray drying unit, drum dryer unit, freeze drying unit, lyophilizing unit, and combinations thereof. In one aspect, the dried solid cell debris portion contains at least about 50% of protein, in one aspect, at least about 60% of protein, in one aspect, at least about 70% of protein, in one aspect, at least about 80% of protein, in another aspect, about 50 to about 90% of protein, in another aspect, about 60 to about 80% of protein, and in still another aspect, about 70 to about 85% of protein.

The protein-containing supernatant contains soluble proteins and amino acids. In one aspect, it contains about 1 to about 25% of protein, in one aspect, about 1 to about 21% of protein, in one aspect, about 1 to about 15% of protein, and in another aspect, about 1.5 to about 15% of protein. Further, the protein-containing supernatant may include less than 5% nucleic acid. In one aspect, the protein-containing supernatant include less than 4% nucleic acid, in another aspect, less than about 3% nucleic acid, in another aspect, less than 2% nucleic acid, in another aspect, less than 1% nucleic acid, in another aspect, nucleic acid is not detectable. In general, the protein-containing supernatant includes ten essential amino acids and several other amino acids.

The protein-containing supernatant may be directly used as or further processed to a nutrient supplement. A dehydration unit may be used to dry the protein-containing supernatant and produce a protein containing supplement, such as protein powder. Suitable dehydration unit includes spray drying unit, drum dryer unit, freeze drying unit, lyophilizing unit, and combinations thereof. Other components, such as moisture and ash, can be further removed to purify the protein containing supplement. The protein containing supplement may be directly used or be blended with other ingredients for making into one or more types of nutrient supplements, such as animal feed, microbial nutrition, and pharmaceutical compositions. In one aspect, the protein containing supplement contains about 60 to about 99 weight percent protein, in another aspect, about 70 to about 95 weight percent protein, in another aspect, about 75 to about 95 weight percent protein, in another aspect, about 80 to 95 weight percent protein, and in another aspect, about 85 to 95 weight percent protein. List of free amino acids and their concentrations in the protein containing supplement is shown as follows:

| Amino Acid | Concentration mg/100 g |
|---|---|
| Aspartic Acid | 230 to 650 |
| Threonine | 50 to 200 |
| Serine | 70 to 300 |
| Glutamine | 10 to 35 |
| Glutamic Acid | 400 to 800 |
| Proline | 60 to 250 |
| Glycine | 200 to 520 |
| Alanine | 230 to 610 |
| Cysteine | 2 to 6 |
| Valine | 50 to 960 |
| Methionine | 80 to 330 |
| Isoleucine | 20 to 500 |
| Leucine | 150 to 550 |
| Tyrosine | 70 to 340 |
| Phenylalanine | 105 to 390 |
| Lysine | 200 to 800 |
| histidine | 10 to 150 |
| Arginine | 110 to 360 |
| Tryptophan | 5 to 70 |
| Asparagine | 5 to 220 |

The protein containing supplement may be used as microbial nutrition to support the growth of microorganisms. Traditionally, microbial growth media contains yeast extract, peptones, and salts. The protein containing supplement may replace part or all of the commercial peptones in the growth media. In this aspect, at least one salt may also be eliminated from the growth media.

The protein-containing supernatant may be delivered to a protein-containing supernatant holding tank and be further processed into an amino acid fertilizer, which can be utilized as a source of nitrogen, carbon, and beneficial metal nutrient elements, such as Co, Fe, Mn, Cu, Mo, Ni, and Zn, for plants. Since the protein-containing supernatant may lack some of the nutrient elements that plants need or have an insufficient amount of some specific nutrient elements, one or more supplements is added to form the amino acid fertilizer. Useful supplements added includes magnesium, calcium, copper, iron, zinc, boron, molybdenum, carbohydrates, sugar, fatty acids, vitamins, and combinations thereof. Further, the protein-containing supernatant may also be concentrated to provide a higher amino acid and nutrient element concentration.

The processed protein-containing supernatant can be directly used as a liquid amino acid fertilizer. In this aspect, the liquid amino acid fertilizer has a free amino acid concentration of about 100 g/L or more, in one aspect, about 150 g/L or more, and in one aspect, about 200 g/L or more. In one aspect, the liquid amino acid fertilizer is a middle element type amino acid fertilizer and contains a concentration of middle element (e.g., calcium and magnesium) of about 30 g/L or more, in one aspect, about 35 g/L or more, and in one aspect, about 40 g/L or more. In another aspect, the liquid amino acid fertilizer is a microelement type amino acid fertilizer and the microelement (e.g., copper, iron, manganese, zinc, boron, and molybdenum) concentration is about 21) g/L or more, in another aspect, about 25 g/L or more, and in still another aspect, about 30 g/L or more.

The processed protein-containing supernatant may also be further dehydrated and processed into a soluble solid amino acid fertilizer. In this aspect, a dehydration unit is used to dry the processed protein-containing supernatant into low moisture soluble solid amino acid fertilizer. Suitable dehydration unit includes spray drying unit, drum dryer unit, freeze drying unit, lyophilizing unit, and combinations thereof. Other components, such as moisture and ash, can be further removed to purify the soluble solid amino acid fertilizer. In this aspect, the soluble solid amino acid fertilizer has a free amino acid concentration of about 10% or more, in one aspect, about 15% or more, in one aspect, about 240 or more, and in one aspect, about 25% or more. In one aspect, the soluble solid amino acid fertilizer is a middle element type amino acid fertilizer and contains a concentration of middle element (e.g., calcium and magnesium) of about 3% or more, in one aspect, about 5% or more, in one aspect about 6.5% or more, and in one aspect, about 8% or more. In another aspect, the soluble solid amino acid fertilizer is a microelement type amino acid fertilizer and the microelement (e.g., copper, iron, manganese, zinc, boron, and molybdenum) concentration is about 2% or more. In another aspect, about 3% or more, in another aspect, about 4% or more, and still in another aspect, about 5% or more.

The protein-containing supernatant may be further separated into fractions rich in certain types of amino acid or individual amino acid. Chromatographic methods based on ionic charge, hydrophobicity, hydrophilicity, or the size of the amino acid can be used for such separation. Thus, a certain chemical element can be removed from the protein-containing supernatant by separating and removing the element binding amino acid. In one aspect, the chemical element to be removed is selenium. In this aspect, selenium is bound with cysteine and methionine. A low selenium protein supplement is produced after the two amino acids are removed. In one aspect, the low selenium protein supplement contains 5 ppm of selenium or less, in one aspect, 4 ppm or less, in one aspect, 3 ppm or less, in one aspect, 2 ppm or less, in one aspect, 1 ppm or less, and m one aspect, 0.5 ppm or less. The removed selenium containing amino acids can further be used as a selenium rich feed additive. The selenium rich feed additive may be further blended with animal feeds for animal and pet consumption. In one aspect, the selenium rich feed additive contains about 5% or more selenium, in one aspect, about 10% or more selenium, in one aspect, about 20% or more selenium, in another aspect, about 30% or more selenium, and in still another aspect, about 40% or more selenium.

### Bacterial Fermentation Systems for Processing Microbial Biomass to Produce Nutrient Supplement

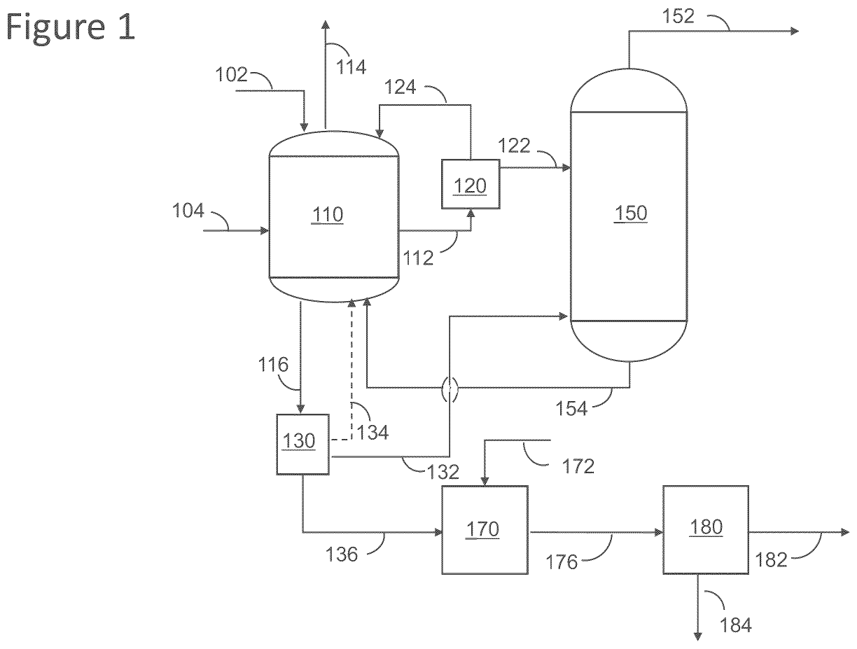

FIG. 1 illustrates a schematic of a system for producing a protein-containing supernatant and one or more oxygenated hydrocarbon compounds from a fermentation process using one species of acetogenic bacteria. The system includes a fermentation vessel 110, a first cell separator 120, a second cell separator 130, a distillation chamber 150, a digestion tank 170, and a fractionator 180.

Two or more inlet lines. e.g., an inlet line 102 and an inlet line 104, are connected to the fermentation vessel 110. The inlet line 102 can be used for delivery of fermentation medium and the inlet line 104 can be used for delivery of Cl-containing gaseous substrate. Vent gas from the fermentation vessel 110 is released through a gas outlet line 114. A first fermentation liquid broth from the fcrmcntation vessel 110 is delivered to the first cell separator 120 through an outlet line 112. In the first cell separator 120, the first fermentation liquid broth is separated into a first cell-free permeate and a first cell-containing suspension. The first cell-free permeate is then delivered to the distillation chamber 150 through an outlet line 122 to produce oxygenated hydrocarbon compound and at least a portion of the first cell-containing suspension is recycled back to the fermentation vessel 110 through an outlet line 124 to maintain and control cell concentration of the fermentation liquid broth. A second fermentation liquid broth from the fermentation vessel 110 is delivered to the second cell separator 130 through an outlet line 116. In the second cell separator 130, the second fermentation liquid broth is separated into a second cell-free permeate and a second cell-containing suspension. The second cell-free permeate is then delivered to the distillation chamber 150 through an outlet line 132 to produce oxygenated hydrocarbon compound and the second cell-containing suspension is delivered to a digestion tank 170 through an outlet line 136 for enzyme hydrolysis. Optionally, at least a portion of the second cell-containing suspension is recycled back to the fermentation vessel 110 through an outlet line 134 to maintain and control cell concentration of the fermentation liquid broth.

The distillation chamber 150 is capable of receiving and processing the cell-free permeates into one or more high-quality oxygenated hydrocarbon compound products (e.g., 95% w/w or higher concentration and/or anhydrous form of ethanol, butanol). The oxygenated hydrocarbon compound product is sent out from the distillation chamber 150 through an outlet line 152. At least a portion of the distillation bottom is recycled back to the fermentation vessel 110 through an outlet line 154.

The digestion tank 170 receives at least a portion of the second cell-containing suspension and produces a hydrolyzed lysate. Hydrolase enzyme is injected into the digestion tank 170 through an inlet line 172. In one aspect, the digestion tank 170 has a temperature control unit to adjust, control and maintain its temperature. In another aspect, the digestion tank 170 has an agitator to agitate the received cell-containing suspension to facilitate enzyme hydrolysis. In another aspect, the digestion tank 170 has a pH probe and a base addition pump to adjust and control pH. In still another aspect, the digestion tank 170 has both a temperature control unit, an agitator, a pH probe, and a base addition pump. The hydrolyzed lysate is then delivered to the fractionator 180 through an outlet line 176. In the fractionator 180, the hydrolyzed lysate is further fractionated into a protein-containing supernatant and a solid cell debris portion. The protein-containing supernatant is delivered out through an outlet line 182 and the solid cell debris portion is delivered out through an outlet line 184.

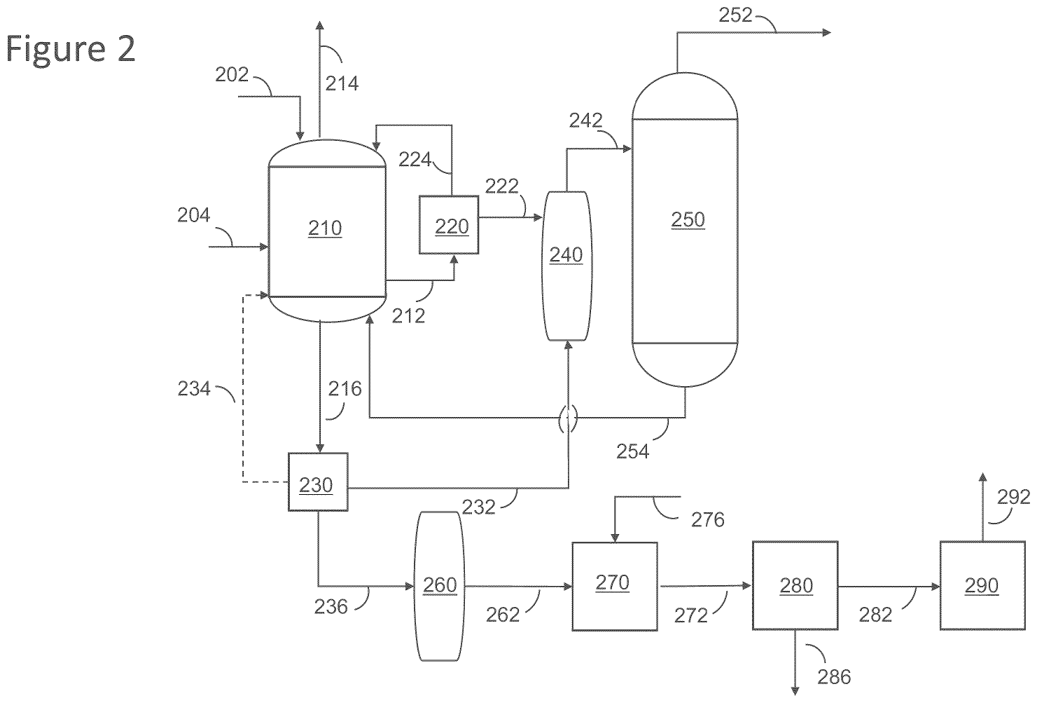

FIG. 2 illustrates a schematic of a system for producing a protein powder and one or more oxygenated hydrocarbon compounds from a fermentation process using one species of acetogenic bacteria. The system includes a fermentation vessel 210, a first cell separator 220, a second cell separator 230, a permeate holding tank 241, a distillation chamber 250, a cell-containing suspension holding tank 260, a digestion tank 271, a fractionator 280, and a dehydration unit 290.

Two or more inlet lines, e.g., an inlet line 202 and an inlet line 204, are connected to the fermentation vessel 210. The inlet line 202 can be used for delivery of fermentation medium and the inlet line 204 can be used tor delivery of Cl-containing gaseous substrate. Vent gas from the fermentation vessel 210 is released through a gas outlet line 214. A first fermentation liquid broth from the fermentation vessel 210 is delivered to the first cell separator 220 through an outlet line 212. In the first cell separator 220, the first fermentation liquid broth is separated into a first cell-free permeate and a first cell-containing suspension. The first cell-free permeate is then sent to the permeate holding tank 240 through an outlet line 222 and at least a portion of the first cell-containing suspension is recycled back to the fermentation vessel 210 through an outlet line 224 to maintain and control cell concentration of the fermentation liquid broth. A second fermentation liquid broth from the fermentation vessel 210 is delivered to the second cell separator 230 through an outlet line 216. In the second cell separator 230, the second fermentation liquid broth is separated into a second cell-free permeate and a second cell-containing suspension. The second cell-free permeate is then delivered to the permeate holding tank 210 through an outlet line 232 and the second cell-containing suspension is delivered to the cell-containing suspension holding tank 260 through an outlet line 236. Optionally, at least a portion of the second cell-containing suspension is recycled back to the fermentation vessel 210 through an outlet line 234 to maintain and control cell concentration of the fermentation liquid broth.

The permeate holding tank 240 receives both the first cell-free permeate and the second cell-free permeate and controls the permeate flow rate to the distillation chamber 250. The mixed cell-free permeate is then sent to the distillation chamber 250 through an outlet line 242. The distillation chamber 250 is capable of processing the cell-free permeate into one or more high-quality oxygenated hydrocarbon compound products (e.g., 95% w/w or higher concentration and/or anhydrous form of ethanol, butanol). The oxygenated hydrocarbon compound product is sent out from the distillation chamber 250 through an outlet line 252. At least a portion of the distillation bottom is recycled back to the fermentation vessel 210 through an outlet line 254.

The cell-containing suspension holding tank 260 receives at least a portion of the second cell-containing suspension and delivers the received cell-containing suspension to the digestion tank 270 through an outlet line 262 at a desired flow rate. The digestion tank 270 is capable of processing the cell-containing suspension into hydrolyzed lysate. Hydrolase enzyme is injected into the digestion tank 270 through an inlet line 276. In one aspect, the digestion tank 270 has a temperature control unit to adjust, control and maintain its temperature. In another aspect, the digestion tank 270 has an agitator to agitate the received cell-containing suspension to facilitate enzyme hydrolysis. In another aspect, the digestion tank 270 has a pH probe and a base addition pump to adjust and control pH. In still another aspect, the digestion tank 270 has both a temperature control unit, an agitator, a pH probe, and a base addition pump. The hydrolyzed lysate is then delivered to the fractionator 280 through an outlet line 272. In the fractionator 280, the hydrolyzed lysate is further fractionated into a protein-containing supernatant and a solid cell debris portion. The protein-containing supernatant is delivered to the dehydration unit 290 through an outlet line 282 and the solid cell debris portion is delivered out through an outlet line 286. The dehydration unit 290 then processes the received protein-containing supernatant into a protein containing supplement, such as protein powder. The protein containing supplement is delivered out through an outlet line 292.

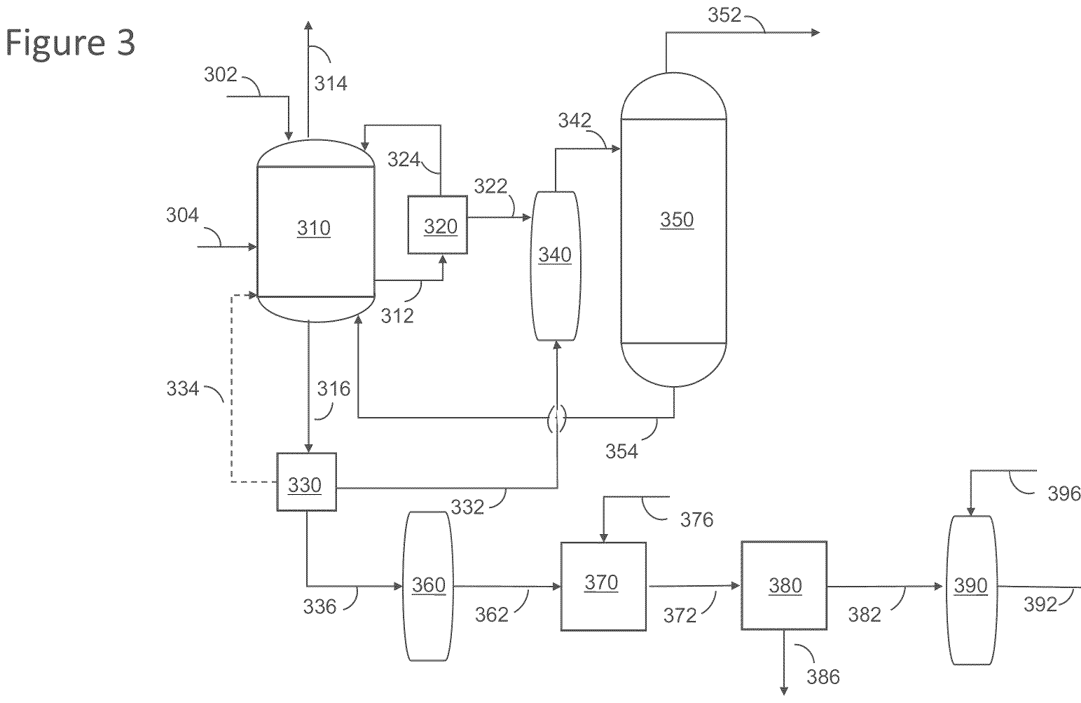

FIG. 3 illustrates a schematic of a system for producing an amino acid fertilizer and one or more oxygenated hydrocarbon compounds from a fermentation process using one species of acetogenic bacteria. The system includes a fermentation vessel 310, a first cell separator 320, a second cell separator 330, a permeate holding tank 340, a distillation chamber 350, a cell-containing suspension holding tank 360, a digestion tank 370, a fractionator 380, and a protein-containing supernatant holding tank 390.

Two or more inlet lines, e.g. an inlet line 302 and an inlet line 304, are connected to the fermentation vessel 310. The inlet line 302 can be used for delivery of fermentation medium and the inlet line 304 can be used for delivery of Cl-containing gaseous substrate. Vent gas from the fermentation vessel 310 is released through a gas outlet line 314. A first fermentation liquid broth from the fermentation vessel 310 is delivered to the first cell separator 320 through an outlet line 312. In the first cell separator 320, the first fermentation liquid broth is separated into a first cell-free permeate and a first cell-containing suspension. The first cell-free permeate is then sent to the permeate holding tank 340 through an outlet line 322 and at least a portion of the first cell-containing suspension is recycled back to the fermentation vessel 310 through an outlet line 324 to maintain and control cell concentration of the fermentation liquid broth. A second fermentation liquid broth from the fermentation vessel 310 is delivered to the second cell separator 330 through an outlet line 316. In the second cell separator 330, the second fermentation liquid broth is separated into a second cell-free permeate and a second cell-containing suspension. The second cell-free permeate is then delivered to the permeate holding tank 340 through an outlet line 332 and the second cell-containing suspension is delivered to the cell-containing suspension holding tank 360 through an outlet line 336. Optionally, at least a portion of the second cell-containing suspension is recycled back to the fermentation vessel 310 through an outlet line 334 to maintain and control cell concentration of the fermentation liquid broth.

The permeate holding tank 340 receives both the first cell-free permeate and the second cell-free permeate and controls the permeate flow rate to the distillation chamber 350. The mixed cell-free permeate is then sent to the distillation chamber 35) through an outlet line 342. The distillation chamber 350 is capable of processing the cell-free permeate into one or more high-quality oxygenated hydrocarbon compound products (e.g., 95% w/w or higher concentration and/or anhydrous form of ethanol, butanol). The oxygenated hydrocarbon compound product is sent out from the distillation chamber 350 through an outlet line 352. At least a portion of the distillation bottom is recycled back to the fermentation vessel 310 through an outlet line 354.

The cell-containing suspension holding tank 360 receives at least a portion of the second cell-containing suspension and delivers the received cell-containing suspension to the digestion tank 370 through an outlet line 362 at a desired flow rate. The digestion tank 370 is capable of processing the cell-containing suspension into hydrolyzed lysate. Hydrolase enzyme is injected into the digestion tank 370 through an inlet line 376. In one aspect, the digestion tank 370 has a temperature control unit to adjust, control and maintain its temperature. In another aspect, the digestion tank 370 has an agitator to agitate the received cell-containing suspension to facilitate enzyme hydrolysis. In another aspect, the digestion tank 370 has a pH probe and a base addition pump to adjust and control pH. In still another aspect, the digestion tank 370 has both a temperature control unit, an agitator, a pH probe, and a base addition pump. The hydrolyzed lysate is then delivered to the fractionator 380 through an outlet line 372. In the fractionator 380, the hydrolyzed lysate is further fractionated into a protein-containing supernatant and a solid cell debris portion. The protein-containing supernatant is delivered to the protein-containing supernatant holding tank 390 through an outlet line 382 and the solid cell debris portion is delivered out through an outlet line 386. The protein-containing supernatant holding tank 390 receives one or more supplements from an inlet line 396 and processes the one or more supplements and the protein-containing supernatant into an amino acid fertilizer. In one aspect, the amino acid fertilizer is delivered out through an outlet line 392 and is directly used as a liquid amino acid fertilizer. In another aspect, the amino acid fertilizer can be delivered to a dehydration unit (not shown on the figure) and processed into a soluble solid amino acid fertilizer.

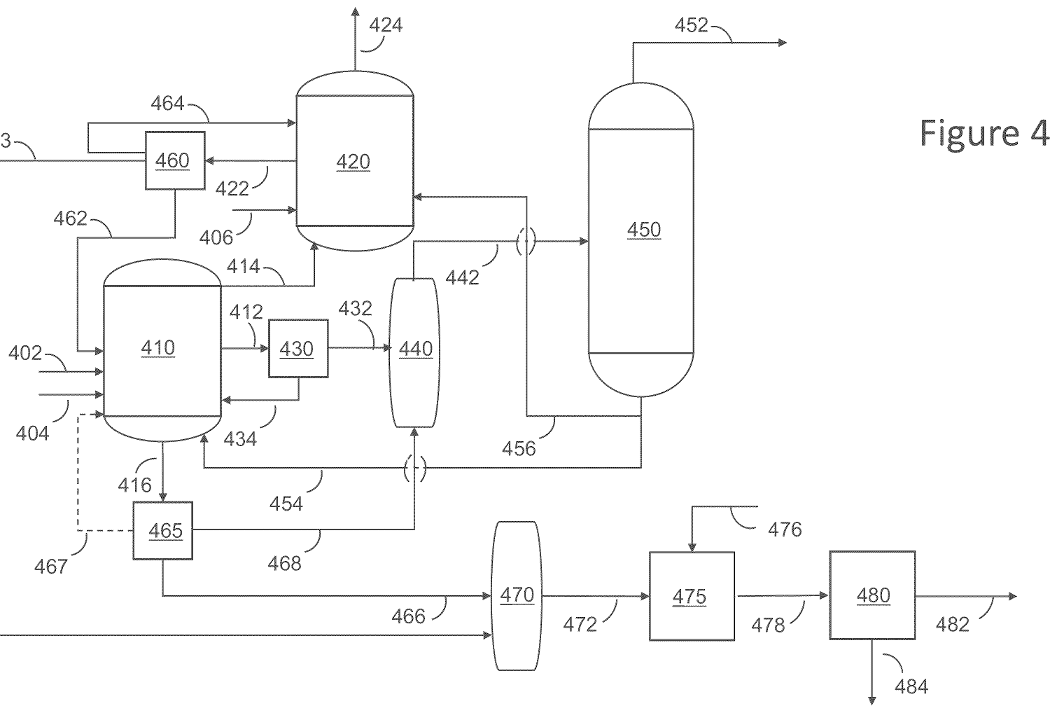

FIG. 4 illustrates a schematic of a system for producing a protein-containing supernatant and one or more oxygenated hydrocarbon compounds from a multi-vessel fermentation process using two or more species of acetogenic bacteria. The system includes a first fermentation vessel 4111, a second fermentation vessel 420, a first cell separator 434, a permeate holding tank 440, a distillation chamber 450, a second cell separator 460, a third cell separator 465, a cell-containing suspension holding tank 470, a digestion tank 475, and a fractionator 480.

Two or more inlet lines. e.g., an inlet line 402 and an inlet line 404, are connected to the first fermentation vessel 410. The inlet line 402 can be used for delivery of fermentation medium and the inlet line 404 can be used for delivery of CO-containing gaseous substrate. In this aspect, the first fermentation vessel 410 contains a first species of acetogenic bacteria performing CO bioconversion and producing a first oxygenated hydrocarbon compound. A first fermentation liquid broth from the first fermentation vessel 410 is delivered to the first cell separator 430 through an outlet line 412. In the first cell separator 430, the first fermentation liquid broth is separated into a first cell-free permeate contains the first oxygenated hydrocarbon compound and a first cell-containing suspension with the cells of the first species of acetogenic bacteria. The first cell-free permeate is then delivered to the permeate holding tank 440 through an outlet line 432 and at least a portion of the first cell-containing suspension is recycled back to the first fermentation vessel 410 through an outlet line 434 to maintain and control cell concentration of the fermentation liquid broth in the first fermentation vessel 410.

Vent gas from the first fermentation vessel 410 contains CO₂ and at least a portion of the vent gas is sent to the second fermentation vessel 420 as a CO₂-containing gaseous substrate through an outlet line 414. The vent gas from the first fermentation vessel 410 may be blended with other gas streams to form a desired CM-containing gaseous substrate with appropriate CO₂ to H₂ ratio before enters the second fermentation vessel 420. One or more inlet lines, e.g., an inlet line 406, is connected to the second fermentation vessel 420. The inlet line 406 can be used for delivery of fermentation medium. In this aspect, second fermentation vessel 420 contains a second species of acetogenic bacteria performing CO bioconversion and producing a second oxygenated hydrocarbon compound. Vent gas from the second fermentation vessel 4211 is released though a gas outlet line 424. A second fermentation liquid broth from the second fermentation vessel 420 is delivered to the second cell separator 460 through an outlet line 422. In the second cell separator 460, the second fermentation liquid broth is separated into a second cell-free permeate contains the second oxygenated hydrocarbon compound and a second cell-containing suspension with the cells of the second species of acetogenic bacteria. At least a portion of the second cell-free permeate contains the second oxygenated hydrocarbon compound is sent to the first fermentation vessel 410 through an outlet line 462. In this aspect, the first species of acetogenic bacteria in the first fermentation vessel 410 may convert at least a portion of the second oxygenated hydrocarbon compound the first fermentation vessel 411 received into the first oxygenated hydrocarbon compound and/or other oxygenated hydrocarbon compounds. Further, a first portion of the second cell-containing suspension is recycled back to the second fermentation vessel 420 through an outlet line 464 to maintain and control cell concentration of the fermentation liquid broth in the second fermentation vessel 420. A second portion of the second cell-containing suspension is sent to the cell-containing suspension holding tank 470 through an outlet line 463.

A third fermentation liquid broth from the first fermentation vessel 410 is purged to the third cell separator 465 through an outlet line 416 and is separated into a third cell-free permeate contains the first oxygenated hydrocarbon compound and a third cell-containing suspension with the cells of the first species of acetogenic bacteria. The third cell-free permeate is then delivered to the permeate holding tank 440 through an outlet line 468 and the third cell-containing suspension is delivered to the cell-containing suspension holding tank 470 through an outlet line 466. Optionally, at least a portion of the third cell-containing suspension is recycled back to the first fermentation vessel 410 through an outlet line 467 to maintain and control cell concentration of the fermentation liquid broth in the first fermentation vessel 410.

The permeate holding tank 440 receives both the first cell-free permeate and the third cell-free permeate and controls the permeate flow rate to the distillation chamber 450. The mixed cell-free permeate is then sent to the distillation chamber 450 through an outlet line 442. In one aspect, both the first cell-free permeate and the third cell-free permeate contain the first oxygenated hydrocarbon compound and the first oxygenated hydrocarbon compound is the target oxygenated hydrocarbon compound product. The distillation chamber 450 is capable of processing the cell-free permeate into one or more high-quality oxygenated hydrocarbon compound products (e.g., 95% w/w or higher concentration and/or anhydrous form of ethanol, butanol). The oxygenated hydrocarbon compound product is sent out from the distillation chamber 450 through an outlet line 452. At least a first portion of the distillation bottom is recycled back to the first fermentation vessel 410 through an outlet line 454 and at least a second portion of the distillation bottom is recycled back to the second fermentation vessel 420 through an outlet line 456.

The cell-containing suspension holding tank 470 receives at least a portion of the second cell-containing suspension with cells of the second species of acetogenic bacteria and at least a portion of the third cell-containing suspension with cells of the first species of acetogenic bacteria. In one aspect, a mixed cell-containing suspension with cells of two or more species of acetogenic bacteria is formed within the cell-containing suspension holding tank 470. In another aspect, the mixed cell-containing suspension with cells of two or more species of acetogenic bacteria is formed before entering the cell-containing suspension holding tank 470. The mixed cell-containing suspension is delivered to the digestion tank 475 through an outlet line 472 at a desired flow rate. The digestion tank 475 receives the mixed cell-containing suspension and produces a hydrolyzed lysate. Hydrolase enzyme is injected into the digestion tank 475 through an inlet line 476. In one aspect, the digestion tank 475 has a temperature control unit to adjust, control and maintain its temperature. In another aspect, the digestion tank 475 has an agitator to agitate the received cell-containing suspension to facilitate enzyme hydrolysis. In another aspect, the digestion tank 475 has a pH probe and a base addition pump to ad just and control pH. In still another aspect, the digestion tank 475 has both a temperature control unit, an agitator, a pH probe, and a base addition pump. The hydrolyzed lysate is then delivered to the fractionator 480 through an outlet line 478. In the fractionator 480, the hydrolyzed lysate is further fractionated into a protein-containing supernatant and a solid cell debris portion. The protein-containing supernatant is delivered out through an outlet line 482 and the solid cell debris portion is delivered out through an outlet line 484.

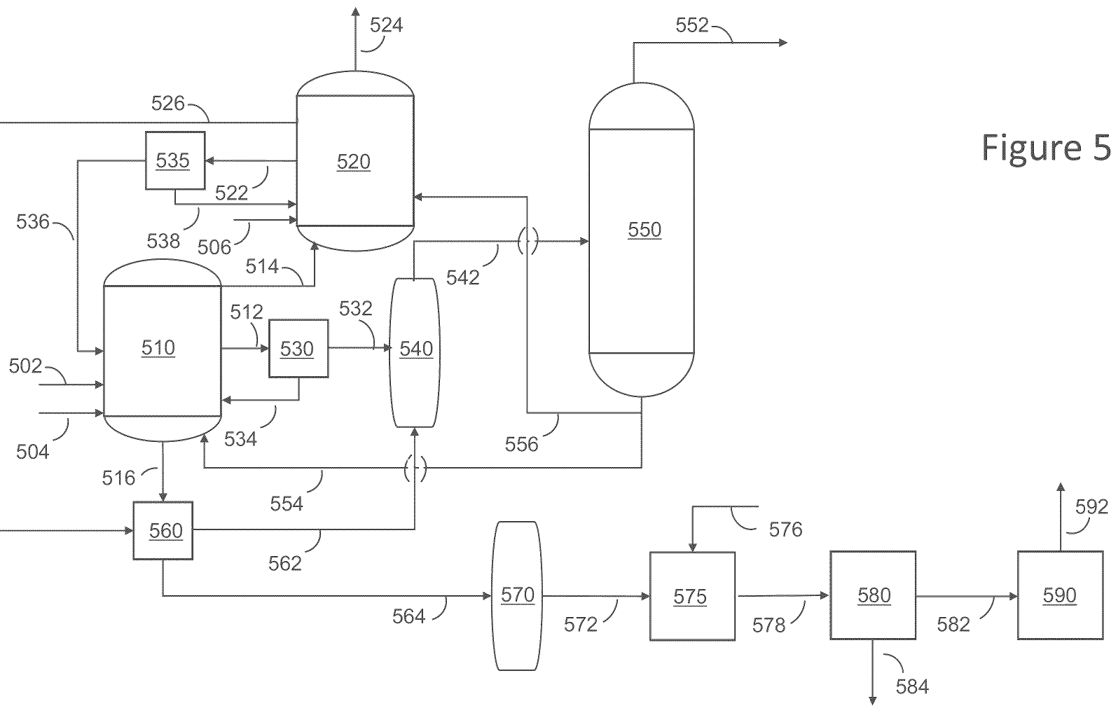

FIG. 5 illustrates a schematic of a system for producing a protein powder and one or more oxygenated hydrocarbon compounds from a multi-vessel fermentation process using two or more species of acetogenic bacteria. The system includes a first fermentation vessel 50, a second fermentation vessel 520, a first cell separator 530, a second cell separator 535, a permeate holding tank 540, a distillation chamber 550, a third cell separator 560, a cell-containing suspension holding tank 570, a digestion tank 575, a fractionator 580, and a dehydration unit 590.

Two or more inlet lines. e.g., an inlet line 502 and an inlet line 504, are connected to the first fermentation vessel 510. The inlet line 502 can be used for delivery of fermentation medium and the inlet line 504 can be used for delivery of CO-containing gaseous substrate. In this aspect, the first fermentation vessel 510 contains a first species of acetogenic bacteria performing CO bioconversion and producing a first oxygenated hydrocarbon compound. A first fermentation liquid broth from the first fermentation vessel 510 is delivered to the first cell separator 530 through an outlet line 512. In the first cell separator 530, the first fermentation liquid broth is separated into a first cell-free permeate contains the first oxygenated hydrocarbon compound and a first cell-containing suspension with the cells of the first species of acetogenic bacteria. The first cell-free permeate is then delivered to the permeate holding tank 540 through an outlet line 532 and at least a portion of the first cell-containing suspension is recycled back to the first fermentation vessel 510 through an outlet line 534 to maintain and control cell concentration of the fermentation liquid broth in the first fermentation vessel 510.

Vent gas from the first fermentation vessel 510 contains CO₂ and at least a portion of the vent gas is sent to the second fermentation vessel 520 as a CO₂-containing gaseous substrate through an outlet line 514. The vent gas from the first fermentation vessel 510 may be blended with other gas streams to form a desired CO-containing gaseous substrate with appropriate CO₂ to H₂ ratio before enters the second fermentation vessel 520. One or more inlet lines, e.g., an inlet line 506, is connected to the second fermentation vessel 520. The inlet line 506 can be used for delivery of fermentation medium. In this aspect, second fermentation vessel 520 contains a second species of acetogenic bacteria performing CO₂ bioconversion and producing a second oxygenated hydrocarbon compound. Vent gas from the second fermentation vessel 520 is released though a gas outlet line 524. A second fermentation liquid broth from the second fermentation vessel 520 is delivered to the second cell separator 535 through an outlet line 522. In the second cell separator 535, the second fermentation liquid broth is separated into a second cell-free permeate contains the second oxygenated hydrocarbon compound and a second cell-containing suspension with the cells of the second species of acetogenic bacteria. At least a portion of the second cell-free permeate contains the second oxygenated hydrocarbon compound is sent to the first fermentation vessel 510 through an outlet line 536. In this aspect, the first species of acetogenic bacteria in the first fermentation vessel 510 may convert at least a portion of the second oxygenated hydrocarbon compound the first fermentation vessel 510 received into the first oxygenated hydrocarbon compound and/or other oxygenated hydrocarbon compounds. Further, at least a portion of the second cell-containing suspension is recycled back to the second fermentation vessel 520 through an outlet line 538 to maintain and control cell concentration of the fermentation liquid broth in the second fermentation vessel 520.

A third fermentation liquid broth from the second fermentation vessel 520 is delivered to the third cell separator 560 through an outlet line 526 and a fourth fermentation liquid broth from the first fermentation vessel 510 is delivered to the third cell separator 560 through an outlet line 516. The fermentation liquid broths from different fermentation vessels are mixed and separated into a third cell-free permeate contains both the first oxygenated hydrocarbon compound and the second oxygenated hydrocarbon compound and a mixed third cell-containing suspension with both the cells of the first species of acetogenic bacteria and the cells of the second species of anerobic bacteria. The third cell-free permeate is then delivered to the permeate holding tank 540 through an outlet line 562 and the mixed cell-containing suspension is delivered to the cell-containing suspension holding tank 570 through an outlet line 564.

The permeate holding tank 540 receives both the first cell-free permeate and the third cell-free permeate and controls the permeate flow rate to the distillation chamber 550. The mixed cell-free permeate is then sent to the distillation chamber 550 through an outlet line 542. In one aspect, both the first cell-free permeate and the third cell-free permeate contain the first oxygenated hydrocarbon compound and the second oxygenated hydrocarbon compound and the first oxygenated hydrocarbon compound is the target oxygenated hydrocarbon compound product. In another aspect, the mixed cell-free permeate contains the first oxygenated hydrocarbon compound and the second oxygenated hydrocarbon compound and both oxygenated hydrocarbon compounds are the target oxygenated hydrocarbon compound products. The distillation chamber 550 is capable of processing the cell-free permeate into one or more high-quality oxygenated hydrocarbon compound products (e.g., 95% w/w or higher concentration and/or anhydrous form of ethanol, butanol). The oxygenated hydrocarbon compound product is sent out from the distillation chamber 550 through an outlet line 552. At least a first portion of the distillation bottom is recycled back to the first fermentation vessel 510 through an outlet line 554 and at least a second portion of the distillation bottom is recycled back to the second fermentation vessel 520 through an outlet line 556.

The mixed cell-containing suspension is delivered from the cell-containing holding tank 570 to the digestion tank 575 through an outlet line 572 at a desired flow rate. The digestion tank 575 is capable of processing the cell-containing suspension into hydrolyzed lysate. Hydrolase enzyme is injected into the digestion tank 575 through an inlet line 576. In one aspect, the digestion tank 575 has a temperature control unit to adjust, control and maintain its temperature. In another aspect, the digestion tank 575 has an agitator to agitate the received cell-containing suspension to facilitate enzyme hydrolysis. In another aspect, the digestion tank 575 has a pH probe and a base addition pump to adjust and control pH. In still another aspect, the digestion tank 575 has both a temperature control unit, an agitator, a pH probe, and a base addition pump. The hydrolyzed lysate is then delivered to the fractionator 580 through an outlet line 578. In the fractionator 580, the hydrolyzed lysate is further fractionated into a protein-containing supernatant and a solid cell debris portion. The protein-containing supernatant is delivered to the dehydration unit 590 through an outlet line 582 and the solid cell debris portion is delivered out through an outlet line 584. The dehydration unit 590 then processes the received protein-containing supernatant into a protein containing supplement, such as protein powder. The protein containing supplement is delivered out through an outlet line 592.

### EXAMPLES

The follow mg examples further illustrate the disclosure and should not be construed to limit its scope.

### Example 1: Continuous Bacterial Fermentation Process with Clostridium ljungdahlii

A stirred tank 2 L reactor containing a suitable medium was inoculated with 0.5 g/L of active Clostridium ljungdahlii. Synthesis gas containing 35% CO, 30% CO₂, 22% H₂, and 13% N₂ was continuously introduced into the reactor. During inoculation, the reactor's agitator was on and a cell recycle system was attached to the reactor. Gas and liquid samples taken from the reactor at every 1 to 4-hour materials were analyzed for consumption or production of various gas components, broth acetic acid concentration, broth ethanol concentration and the optical density of the culture. Also, the composition of the feed-gas was measured daily and the flow to the reactor was maintained at required gas flow rates by using a mass flow controller. After inoculation, cell mass increased with time and reached 3.73 g/L through cell purge. The reactor is then maintained at a steady state at 11 to 13 g/L ethanol concentration and 1.2 to 2.8 g/L acetate with a cell retention time of 31.7 hr and liquid retention time of 25 hr. The average rate of base (NaOH) was 0.2 mil/mi to maintain pH at 4.5.

During the steady state, the following conversions were achieved:
CO: 85% to 95%
H₂: 35% to 50%
Ethanol productivity: 25 g ethanol/L culture/day
Specific ethanol productivity: 6.7 g ethanol day/gram of cells

Fermentation broth samples 1a, 1b, 1c, 1d and 1e were taken during the steady state of the fermentation process.

### Example 2a: Continuous Bacterial Fermentation Process with Acetobacterium woodii

A stirred tank 2 L reactor containing a suitable medium was inoculated with 0.5 g/L of active Acetobacterium woodii. Synthesis gas containing 8% CO, 25% CO₂, 62% H₂, and 5% N₂ was continuously introduced into the reactor. During inoculation, the reactor's agitation rate was on and a cell recycle system was attached to the reactor. Gas and liquid samples taken from the reactor at every 1 to 4-hour intervals were analyzed for consumption or production of various gas components, broth acetic acid concentration, broth ethanol concentration and the optical density of the culture. Also, the composition of the feed-gas was measured daily and the flow to the reactor was maintained at required gas flow rates by using a mass flow controller. After inoculation, cell mass increased with time and maintained at 3 g/L through cell purge. Acetic acid concentration of the fermentation broth was maintained at 8 g/L throughout the steady state. The average rate of base (NaOH) was 2.0 ml/min to maintain pH at 6.

During the steady state, the following conversions were achieved:
CO₂: 45% to 95%
H₂: 39% to 85%
Acetic acid productivity: 38.4 g acetic acid/L culture/day
Specific acetic acid productivity: 12.3 g acetic acid/day/gram of cells

Fermentation broth sample 2a was taken during the steady state of the fermentation process.

### Example 2b: Continuous Bacterial Fermentation Process with Acetobacterium woodii

A stirred tank 60 L reactor containing a suitable medium was inoculated with 0.3 g/L of active Acetobacterium woodii. Synthesis gas containing 1.4% CO, 26% CO₂, 58% H₂ and 14.6% N₂ was continuously introduced into the reactor. During inoculation, the reactor's agitator was on and a cell recycle system was attached to the reactor. Gas and liquid samples taken from the reactor at every 1 to 4-hour intervals were analyzed for consumption or production of various gas components, broth acetic acid concentration, broth ethanol concentration and the optical density of the culture. Also, the composition of the feed-gas was measured daily and the flow to the reactor was maintained at required gas flow rates by using a mass flow controller. After inoculation, cell mass increased with time and maintained at 6 g/L through cell purge. Acetic acid concentration of the fermentation broth was maintained at 8 g/Lt throughout the steady state. pH was maintained at 6 during inoculation and gradually increased after steady state through adding the base NH₄OH.

During the steady state, the following conversions were achieved:
CO₂: 55% to 98%
H₂: 50% to 00%
Acetic acid productivity: 65.5 g acetic acid/L culture/day
Specific acetic acid productivity: 32 g acetic acid/day/gram of cells

Fermentation broth sample 2b was taken during the steady state of the fermentation process.

### Example 3: Protein Powder Recovery from Microbial Biomass

Seven samples of cell mass were acquired from functioning fermentation process as described in Example 1, 2a and 2b. For each sample, a cell-containing suspension was separated from the fermentation broth and concentrated to 120 g/L dry cell weight through a centrifuge at 6,000 rpm for 10 minutes with a temperature of 4° C. The cell-containing suspension was then added into a mixing container and diluted with deionized water with a temperature of 25° C. A solution of 4 g NaOH per 25 ml of deionized water was added to adjust the pH of the diluted cell-containing suspension to 8.2. Alcalase was added to the cell-containing suspension at pH 8.2. The container was heated in a 60° C. incubator shaker with agitation at 65 rpm during the hydrolysis reaction.

The duration of the hydrolysis reaction of the samples varies from 5 to 24 hours. The hydrolyzed lysate was further either centrifuged or filtered into protein-containing supernatant with desired soluble protein.

Centrifugation of hydrolyzed lysate was performed at 47,500×g, for 20 minutes in a temperature of 4° C. When the centrifugation was complete, a protein-containing supernatant with both clear lysate and opaque lysate was collected into a separate container.

Filtration of hydrolyzed lysate was performed with either 500 kDa, 0.1 μm or 0.2 μm filter. The protein-containing supernatant (permeate) was harvested into a separate container. The remaining cell debris may be further processed into other products, such as animal feed or peptone.

The protein-containing supernatant was then spray dried. Conditions of spray drier was set to a temperature of 175° C. with a carrier of compressed air flow of 50 mm volumetric flow rate, a vacuum of 60 mbar and a liquid flow rate of 6 to 10 ml/min. Protein evaluation of the spray dried material was based on Kjeldahl reaction.

Results of each sample are shown in Table 1.

TABLE 1

| | | Enzyme | | | | Protein | |
|---|---|---|---|---|---|---|---|
| | | Concentration | | | Protein | Yield (g) | Protein |
| | | in the | | | Yield (g) | per 100 g | Yield (g) |
| Experiment | | digestion | Hydrolysis | Method of | per 100 g | spray dried | per 100 g |
| No. | Biocatalyst | reaction | Time (hr) | Separation | of cells | supernatant | cell debris |
| 1a | Clostridium | Alcalase | 24 | Centrifuge | 60 | 79 | n/a |
| | ljungdahlii | [0.5%] | | | | | |
| 1b | Clostridium | Alcalase | 5 | Centrifuge | 49 | 78 | n/a |
| | ljungdahlii | [0.5%] | | | | | |
| 1c | Clostridium | Alcalase | 24 | Ultrafilter | 15.6 | 75.6 | 75.1 |
| | ljungdahlii | [0.5%] | | 500 kDa | | | |
| 1d | Clostridium | Alcalase | 24 | Ultrafilter | 20.4 | 73.1 | 72.6 |
| | ljungdahlii | [1.5%] | | 500 kDa | | | |
| 1e | Clostridium | Alcalase | 24 | Ultrafilter | 22.9 | 78.8 | 72.8 |
| | ljungdahlii | [0.5%] | | 0.2 μm | | | |
| 2a | Acetobacterium | Alcalase | 24 | Ultrafilter: | 12.7 | 64.7 | 60.8 |
| | woodii | [0.5%] | | 0.1 μm | | | |
| 2b | Acetobacterium | Alcalase | 24 | Ultrafilter | 30.6 | 66.7 | 64.3 |
| | woodii | [0.5%] | | 0.2 μm | | | |

In experiments 1a and 1b, Clostridium ljungdahlii cell containing fermentation broth samples were taken and the hydrolyzed lysates were separated by centrifugation. Both experiments used 0.5% alcalase concentration in the digestion reaction. Experiment 1a showed a 60% protein yield rate with 24 hours hydrolysis time. Experiment 1b showed a 49% protein yield rate with 5 hours hydrolysis time.

In experiments 1c and 1d, Clostridium ljungdahlii cell containing fermentation broth samples were taken and the hydrolyzed lysates were separated by 5 kDa ultrafilter. Both experiments were performed with 24 hours hydrolysis time. Experiment 1c showed a 15.6% protein yield rate with 0.5% alcalase concentration. Experiment 1d showed a 20.4% protein yield rate with 1.5% alcalase concentration.

In experiments 1c and 1e, Clostridium ljungdahlii cell containing fermentation broth samples were taken and the hydrolyzed lysates were separated by ultrafiltration. Both experiments used 0.5% alcalase concentration with 24 hours hydrolysis time. Experiment 1c was performed with 500 kDa ultrafiltration and showed a 15.6% protein yield rate. Experiment IC was performed with 0.2 pin ultrafilter and showed a 22.9% protein yield rate, which is about 46.79% higher than experiment 1c. Further, the protein yield in spray dried supernatant in experiment 1e is also higher than the rate in experiment 1c.

In experiments 2a and 2b, Acetobacterium woodii cell containing fermentation broth samples were taken and the hydrolyzed lysates were separated by ultrafiltration. Both experiments used 0.5% alcalase concentration with 24 hours hydrolysis time. In experiment 2a, pH in the fermentation reactor was maintained at 6 before the cell containing fermentation broth sample was taken. The separation of the hydrolyzed lysate in experiment 2a was further performed with 0.1 μm ultrafilter and showed a 12.7% protein yield rate. In experiment 2b, pH in the fermentation reactor was maintained at 6 during inoculation and was gradually increased in steady state. The separation of the hydrolyzed lysate in experiment 2b was further performed with 0.2 μm ultrafilter and showed a 30.6% protein yield rate, which is about 140.94% higher than experiment 2a. Further, the protein yield in spray dried supernatant and the protein yield n cell debris in experiment 2b are both higher than experiment 2a. No negative impact on protein yield was observed despite the fermentation broth of experiments 2a and 2b containing higher levels of salts. In this aspect, the process provides a protein containing supplement with 60 to 90 dry weight percent protein when fermentation broth has a sodium ion concentration of about 500 to about 8000 ppm.

### Example 4: Amino Acid Fertilizer Recovery from Microbial Biomass

Sample of cell mass was acquired from functioning fermentation process as described in Example 1. A cell-containing suspension was separated from the fermentation liquid broth and concentrated to 120 g/L dry cell weight. NaOH was added to the cell-containing suspension to adjust its pH to 8.2 and 05% v/v of alcalase was then added. The cell-containing suspension was then heated to 60° C. for 24 hours with a slight agitation of 300 rpm to torn a hydrolyzed lysate. An ultrafiltration unit was used to separate the hydrolyzed lysate into a protein-containing supernatant and a solid cell debris portion. The protein-containing supernatant was then dehydrated into a solid soluble amino acid fertilizer with the addition of supplemental calcium and magnesium.

The solid soluble amino acid fertilizer was rehydrated into a liquid fertilizer by dissolving one part of it solid fertilizer into three pans of water on a volume-to-volume basis. Results of free amino acid analysis in the liquid fertilizer are shown in Table 2

TABLE 2

| Amino Acid | Concentration g/L |
|---|---|
| Aspartic Acid | 20.4 |
| Threonine | 4.8 |
| Serine | 0.5 |
| Glutamine | 1 |
| Glutamic Acid | 25.5 |
| Proline | 6.2 |
| Glycine | 16.4 |
| Alanine | 19.6 |
| Cysteine | 0.2 |
| Valine | 19.28 |
| Methionine | 8 |
| Isoleucine | 20 |
| Leucine | 21 |
| Tyrosine | 7.2 |
| Phenylalanine | 11.3 |
| Lysine | 25.24 |
| histidine | 0.9 |
| Arginine | 9.6 |
| Tryptophan | 1.7 |
| Asparagine | 6.4 |

### Example 5: Protein Containing Supplement as Microbial Nutrition

Sample of cell mass was acquired from functioning fermentation process as described in Example 1. A cell-containing suspension was separated from the fermentation liquid broth and concentrated to 120 g/L dry cell weight. NaOH was added to the cell-containing suspension to adjust its pH to 8.2 and 0.5% v/v of alcalase was then added. The cell-containing suspension was then heated to 60° C. for 24 hours with a slight agitation of 300 rpm to form a hydrolyzed lysate. An ultrafiltration unit was used to separate the hydrolyzed lysate into a protein-containing supernatant and a solid cell debris portion. The protein-containing supernatant was then spray dried to produce a soluble protein containing supplement.

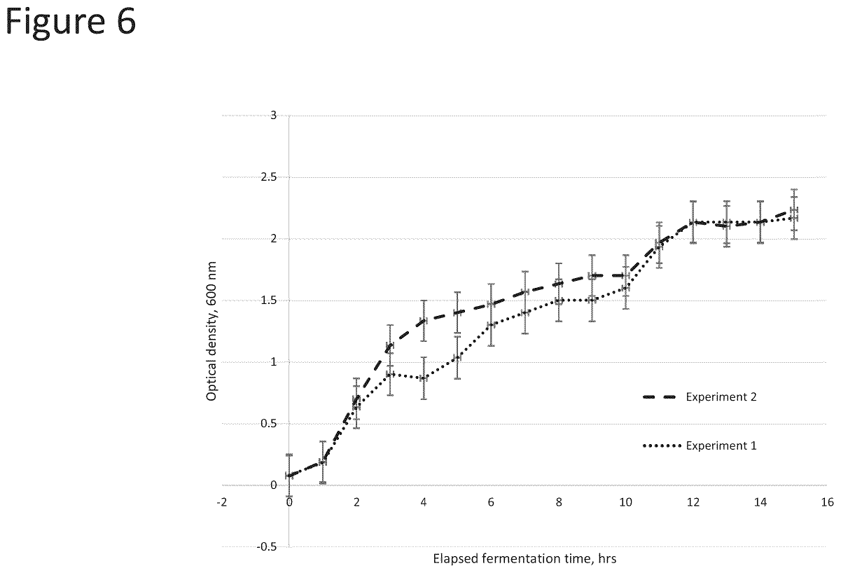

Peptone is required for sufficient growth of Escherichia coli (E. coli). Two shake flask, experiments were conducted to grow E. coli. In experiment 1, 5 g/L yeast extract, 10 g/L commercial peptone, and 5 g/L NaCl were used. In experiment 2, 5 g/L yeast extract and 10 g/L soluble protein containing supplement were used. FIG. 6 illustrates the growth of E. coli in both experiments. The protein containing supplement could replace commercial peptone to support the growth of E. coli. Meanwhile, it is unnecessary to supplement additional salt (NaCl) when the protein containing supplement is in use, which eliminates a media component.

While the disclosure herein disclosed has been described by means of specific embodiments, examples, and applications thereof, other and further variations could be devised without departing from the basic scope of the disclosure set forth in the claims that follow.

What is claimed is:

1. A process for producing a nutrient supplement from an anaerobic fermentation process, the process comprising:
fermenting a gaseous substrate with an acetogenic bacteria in a fermentation vessel;
obtaining from the fermentation vessel an amount of a fermentation liquid broth containing acetogenic bacterial cells;
separating the fermentation liquid broth into a cell-free permeate and a cell-containing suspension;
recovering an oxygenated hydrocarbon compound from the cell-free permeate;
increasing the pH of the cell-containing suspension;
contacting the cell-containing suspension having an increased pH with a hydrolase enzyme;
incubating the cell-containing suspension and the hydrolase enzyme at a temperature of about 50 to about 70° C. for about 3 to about 72 hours to form a hydrolyzed lysate; and
fractionating the hydrolyzed lysate into a protein-containing supernatant and a solid cell debris portion.

2. The process of claim 1 where the gaseous substrate is a CO-containing gas.

3. The fermentation process of claim 2 wherein the CO-containing gaseous substrate has a H₂/CO molar ratio of about 0.2 or more.

4. The fermentation process of claim 1 wherein the acetogenic bacteria is an acetogenic bacteria selected from the group consisting of Clostridium, Acetobacterium, and mixtures thereof.

5. The fermentation process of claim 4 wherein the acetogenic Clostridium bacteria is selected from the group consisting of Clostridium ljungdahlii, Clostridium autoethanogum, Clostridium carboxidivorans, Clostridium drakei, Clostridium coskatii, Clostridium ragsdalei and mixtures thereof.

6. The process of claim 1 wherein the gaseous substrate is a CO₂-containing gas.

7. The fermentation process of claim 6 wherein the CO₂-containing gaseous substrate has a H₂/CO molar ratio of about 4:1 to 1:2.

8. The fermentation process of claim 6 wherein the fermentation broth has a first pH value at inoculation and a second pH value at steady state.

9. The fermentation process of claim 8 wherein the second pH value is higher than the first pH value.

10. The fermentation process of claim 1 wherein the cell-containing suspension has a dry cell weight concentration of about 20 g/liter to about 200 g/liter.

11. The fermentation process of claim 1 wherein the hydrolase enzyme is selected from the group consisting of subtilases, alcalase, serine protease, serine endopeptidase and mixtures thereof.

12. The fermentation process of claim 1, wherein the hydrolyzed lysate is fractionated into the protein-containing supernatant and the solid cell debris portion using centrifugation.

13. The fermentation process of claim 1 wherein the hydrolyzed lysate is fractionated into the protein-containing supernatant and the solid cell debris portion using ultrafiltration.

14. The fermentation process of claim 1 wherein the protein-containing supernatant has a nucleic acid content of less than about 5%.

15. The fermentation process of claim 1 wherein the protein-containing supernatant is dehydrated to provide a protein containing supplement with 60 to about 99 dry weight percent protein.

16. The fermentation process of claim 15 wherein the protein-containing supplement is used as a microbial nutrition in a growth media.

17. The fermentation process of claim 1 wherein at least one supplement is added to the protein-containing supernatant.

18. The fermentation process of claim 16 wherein the supplement comprises magnesium, calcium, copper, iron, zinc, boron, molybdenum, carbohydrates, sugar, fatty acids, vitamins, and mixtures thereof.

19. The fermentation process of claim 1 wherein the protein-containing supernatant is further processed to remove at least two selenium containing amino acids to provide a low selenium protein supplement.

20. The fermentation process of claim 11 wherein the removed at least two selenium containing amino acids are further processed to provide a selenium rich feed additive.

21. A system for producing a nutrient supplement and an oxygenated hydrocarbon compound from a bacterial fermentation process using acetogenic bacteria, the system comprising:
a fermentation vessel containing culture medium and acetogenic bacteria connected to a gas inlet line for flowing a gaseous substrate into the fermentation vessel to ferment the gaseous substrate and the culture medium into a fermentation liquid broth;
one or more cell separators connected to one or more outlets of the fermentation vessel to receive the fermentation broth and produce a cell-free permeate and a cell-containing suspension;
a distillation chamber configured to receive the cell-free permeate and recover the oxygenated hydrocarbon compound;
a digestion tank connected to one or more outlet lines of the one or more cell separators to receive the cell-containing suspension and produce a hydrolyzed lysate, wherein the digestion tank receives one or more hydrolase enzymes and provides an incubation temperature of about 50 to about 70° C.; and
one or more fractionators connected to one or more outlet lines of the digestion tank to receive the hydrolyzed lysate, the one or more fractionators providing a protein-containing supernatant and a cell debris portion.

22. The system of claim 21 wherein the one or more fractionators are selected from the group consisting of centrifugation, filtration, ultrafiltration and combination thereof.

23. The system of claim 21 further comprising a spray dryer.

24. The system of claim 21 wherein two or more cell separators are used.

25. A process for producing a nutrient supplement from an anaerobic fermentation process, the process comprising:
fermenting a gaseous substrate with a first acetogenic bacteria in a first fermentation vessel to produce a first vent gas and a first fermentation liquid broth containing the first acetogenic bacterial cells;
separating the first fermentation liquid broth into a first cell-free permeate and a first cell-containing suspension;
recovering an oxygenated hydrocarbon compound from the first cell-free permeate;
fermenting at least a portion of the first vent gas with a second acetogenic bacteria in a second fermentation vessel to produce a second fermentation liquid broth containing the second acetogenic bacterial cells;
separating the second fermentation liquid broth into a second cell-free permeate and a second cell-containing suspension;
recycling at least a portion of the second cell-free permeate to the first fermentation vessel;
blending at least a portion of the first cell-containing suspension with at least a portion of the second cell-containing suspension to form a mixed cell-containing suspension;
increasing the pH of the mixed cell-containing suspension;
contacting the mixed cell-containing suspension having an increased pH with a hydrolase enzyme;
incubating the mixed cell-containing suspension and enzyme at a temperature of about 50 to about 70° C. for about 3 to about 72 hours to form a hydrolyzed lysate; and
fractionating the hydrolyzed lysate into a protein-containing supernatant and a solid cell debris portion.

26. The process of claim 25 wherein the gaseous substrate is a CO-containing gas.

27. The fermentation process of claim 26 wherein the CO-containing gaseous substrate has a H₂/CO molar ratio of about 0.2 or more.

28. The fermentation process of claim 25 wherein the first acetogenic bacteria is an acetogenic Clostridium.

29. The fermentation process of claim 8 wherein the acetogenic Clostridium is selected from the group consisting of Clostridium ljungdahlii, Clostridium ljungdahlii autoethanogum, Clostridium carboxidivorans, Clostridium drakei, Clostridium coskatii, Clostridium ragsdalei, and mixture thereof.

30. The fermentation process of claim 25 wherein the second acetogenic bacteria is selected from the group consisting of Acetogenium kivui, Acetoanaerobium noterae, Acetobacterium woodii, Alkalibaculum bacchi, Acetobacterium bakii, and mixture thereof.

31. The fermentation process of claim 25 wherein the second fermentation liquid broth has a first pH value at inoculation and a second pH value at steady state.

32. The fermentation process of claim 31 wherein the second pH value is higher than the first pi value.

33. The fermentation process of claim 25 wherein the mixed cell-containing suspension has a dry cell weight concertation of about 20 g/liter to about 200 g/liter.

34. The fermentation process of claim 25 wherein the hydrolases enzyme is selected from the group consisting of subtilases, alcalase, serine protease, serine endopeptidase and mixtures thereof.

35. The fermentation process of claim 25 wherein the hydrolyzed lysate is fractionated into the protein-containing supernatant and the solid cell debris portion using centrifugation.

36. The fermentation process of claim 25 wherein the hydrolyzed lysate is fractionated into the protein-containing supernatant and the solid cell debris portion using ultrafiltration.

37. The fermentation process of claim 25 wherein the protein-containing supernatant has a nucleic acid content of less than about 5%.

38. The fermentation process of claim 25 wherein the protein-containing supernatant is dehydrated to provide a protein containing supplement with 60 to about 99 dry weight percent protein.

39. The fermentation process of claim 25 wherein the protein-containing supplement is used as a microbial nutrition in a growth media.

40. The fermentation process of claim 25 wherein at least one supplement is added to the protein-containing supernatant.

41. The fermentation process of claim 25 wherein the supplement comprises magnesium, calcium, copper, iron, zinc, boron, molybdenum, carbohydrates, sugar, fatty acids, vitamins, and mixtures thereof.

42. The fermentation process of claim 25 wherein the protein-containing supernatant is further processed to remove at least two selenium containing amino acids to provide a low selenium protein supplement.

43. The fermentation process of claim 42 wherein the removed at least two selenium containing amino acids are further processed to provide a selenium rich feed additive.

44. A process for producing a nutrient supplement from an anaerobic fermentation process, the process comprising:
fermenting a gaseous substrate with a first acetogenic bacteria in a first fermentation vessel to produce a first vent gas and a first fermentation liquid broth containing the first acetogenic bacterial cells;
fermenting at least a portion of the first vent gas with a second acetogenic bacteria in a second fermentation vessel to produce a second fermentation liquid broth containing the second acetogenic bacterial cells;
blending at least a portion of the first fermentation liquid broth containing the first acetogenic bacterial cells with at least a portion of the second fermentation liquid broth containing the second acetogenic bacterial cells to form a mixed fermentation liquid broth;
separating the mixed fermentation liquid broth to produce a cell-free permeate and a cell-containing suspension;
recovering an oxygenated hydrocarbon compound from the cell-free permeate;
increasing the pH of the cell-containing suspension;
contacting the cell-containing suspension having an increased pH with a hydrolase enzyme;
incubating the cell-containing suspension and enzyme at a temperature of about 50 to about 70° C. for about 3 to about 72 hours to form a hydrolyzed lysate; and
fractionating the hydrolyzed lysate into a protein-containing supernatant and a solid cell debris portion.

45. A process for producing a nutrient supplement from an anaerobic fermentation process, the process comprising:
fermenting a gaseous substrate with a first acetogenic bacteria in a first fermentation vessel to produce a first vent gas and a first fermentation liquid broth containing the first acetogenic bacterial cells;
fermenting at least a portion of the first vent gas with a second acetogenic bacteria in a second fermentation vessel to produce a second fermentation liquid broth containing the second acetogenic bacterial cells;
separating the first permeation liquid broth into a first cell-free permeate and a first cell-containing suspension;
recovering an oxygenated hydrocarbon compound from the first cell-free permeate;
increasing the pH of the first cell-containing suspension;
contacting the first cell-containing suspension having an increased pH with hydrolase enzyme;
incubating the first cell-containing suspension and enzyme at a temperature of about 50 to about 70 for about 3 to about 72 hours to form a first hydrolyzed lysate;
fractionating the first hydrolyzed lysate into a first protein-containing supernatant and a first solid cell debris portion;
separating the second fermentation liquid broth into a second cell-free permeate and a second cell-containing suspension;
recycling at least a portion of the second cell-free permeate to the first fermentation vessel;
increasing the pH of the second cell-containing suspension;
contacting the second cell-containing suspension having an increased pH with hydrolase enzyme;
incubating the second cell-containing suspension and enzyme at a temperature of about 50 to about 70° C. for about 3 to about 72 hours to form a second hydrolyzed lysate; and
fractionating the second hydrolyzed lysate into a second protein-containing supernatant and a second solid cell debris portion.

46. A process for producing a nutrient supplement from an acetogenic bacteria in an anaerobic fermentation process, the process comprising:
fermenting a gaseous substrate with acetogenic bacteria in a fermentation vessel;
obtaining from the fermentation vessel an amount of a fermentation liquid broth containing acetogenic bacterial cells;
separating the fermentation liquid broth into a cell-free permeate and a cell-containing suspension;
recovering an oxygenated hydrocarbon compound from the cell-free permeate;
increasing the pH of the cell-containing suspension;
contacting the cell-containing suspension having an increased pH with a hydrolase enzyme;
incubating the cell-containing suspension and the hydrolase enzyme at a temperature of about 50 to about 70° C. for about 2 to about 36 hours to form a partial hydrolyzed lysate;
mechanical rupturing the partial hydrolyzed lysate to form a hydrolyzed lysate; and
fractionating the hydrolyzed lysate into a protein-containing supernatant and a solid cell debris portion.
